(19) United States
(12) Patent Application Publication
(10) Pub. No.: US 2025/0282777 A1
(43) Pub. Date: Sep. 11, 2025

(54) Methods of Making Tolebrutinib

(71) Applicant: Principia Biopharma Inc., Bridgewater, NJ (US)

(72) Inventors: David Baltes, Bolbec (FR); Bojan Bohorc, Frankfrut (DE); Peyman Sakhaii, Frankfrut (DE)

(73) Assignee: Principia Biopharma Inc., Bridgewater, NJ (US)

(21) Appl. No.: 19/071,185

(22) Filed: Mar. 5, 2025

Related U.S. Application Data
(60) Provisional application No. 63/561,794, filed on Mar. 6, 2024.
(60) Provisional application No. 63/564,100, filed on Mar. 12, 2024.

(51) Int. Cl. C07D 471/04

(52) U.S. Cl. CPC C07D 471/04

(57) ABSTRACT

Disclosed herein are improved synthetic routes for making (R)-1-(1-acryloylpiperidin-3-yl)-4-amino-3-(4-phenoxyphenyl)-1H-imidazo[4,5-c]pyridin-2(3H)-one (tolebrutinib). Also disclosed herein are novel compounds used in the synthesis of (R)-1-(1-acryloylpiperidin-3-yl)-4-amino-3-(4-phenoxyphenyl)-1H-imidazo[4,5-c]pyridin-2(3H)-one.

### CROSS-REFERENCE TO RELATED APPLICATIONS

This application claims the benefit of priority to U.S. provisional application No. 63/561,794, filed Mar. 6, 2024, and U.S. provisional application No. 63/564,100 filed Mar. 12, 2024, each of which is incorporated by reference herein in its entirety for any purpose.

### FIELD

Disclosed herein are methods for preparing (R)-1-(1-acryloylpiperidin-3-yl)-4-amino-3-(4-phenoxyphenyl)-1H-imidazo[4,5-c]pyridin-2(3H)-one free base (tolebrutinib), also referred to herein as a Compound of Formula (I) having the structure:

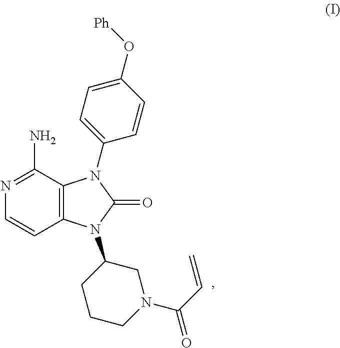 [SMILES: C=CC(=O)N1CCC[C@@H](n2c(=O)n(-c3ccc(Oc4ccccc4)cc3)c3c(N)nccc32)C1]

as well as salt forms thereof, intermediates of (R)-1-(1-acryloylpiperidin-3-yl)-4-amino-3-(4-phenoxyphenyl)-1H-imidazo[4,5-c]pyridin-2(3H)-one, crystalline forms thereof, and methods for preparing same. Compound (1) and its salts and solid state forms thereof are potent Bruton's Tyrosine Kinase (BTK) inhibitors and thus can be useful in the treatment of diseases or disorders resulting from an excess of BTK signaling, for example, a disease selected from an autoimmune disease, an inflammatory disease, or cancer.

### BACKGROUND AND SUMMARY

Compound (1) and a method for preparing it is disclosed in Example 3 of U.S. Pat. No. 9,688,676 B2, at column 62, line 8 to column 65 line 32, and column 67, line 28 to column 69. The disclosed synthesis provides 100 mg of crude Compound (1) that must be purified by column chromatography, resulting in 54.5 mg of purified Compound (1), which is a loss of nearly 50% of the yield. The disclosed synthesis comprises 10 steps, several of which involve undesirable solvents, and can lead to extra waste on a large scale.

One factor in the suitability of a compound as a therapeutic agent is a compound synthesis amendable to large scale manufacturing and isolation with minimal product waste and impurities. This factor is frequently considered when reviewing the suitability of a bench-scale process for making the larger quantities needed for commercial production. Moreover, the environmental impact of the various reagents and conditions needed for large scale manufacturing are an increasingly important factor.

The present disclosure relates to a method of preparing a compound of Formula (I):

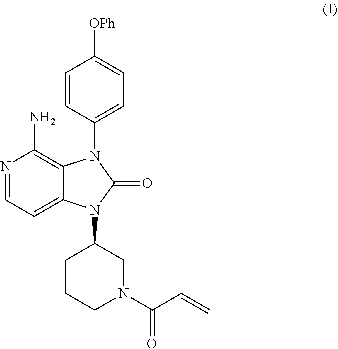 [SMILES: C=CC(=O)N1CCC[C@@H](n2c(=O)n(-c3ccc(Oc4ccccc4)cc3)c3c(N)nccc32)C1]

or a salt thereof, comprising:
reacting a compound of Formula (A-oxalate hydrate):

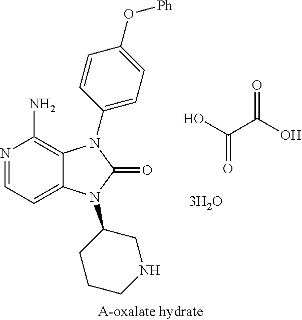 [SMILES: Nc1nccc2c1n(-c1ccc(Oc3ccccc3)cc1)c(=O)n2[C@@H]1CCCNC1 ; O=C(O)C(=O)O]

to form the compound of Formula (I), wherein the reaction comprises the following steps:
   (i) to a solution of the compound Formula (A-oxalate hydrate) adding 3-chloropropanoic acid; and
   (ii) to the mixture of (i), adding 1,8-diazabicyclo[5.4.0]undec-7-ene;
to prepare the compound of Formula (I).

The present disclosure also relates to a method of preparing a compound of Formula (A-oxalate hydrate):

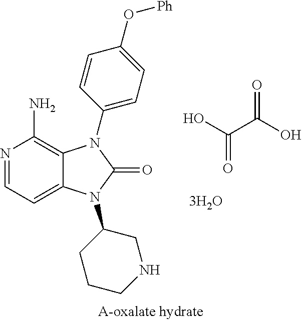 [SMILES: Nc1nccc2c1n(-c1ccc(Oc3ccccc3)cc1)c(=O)n2[C@@H]1CCCNC1 ; O=C(O)C(=O)O]

comprising:
reacting a compound of Formula (A):

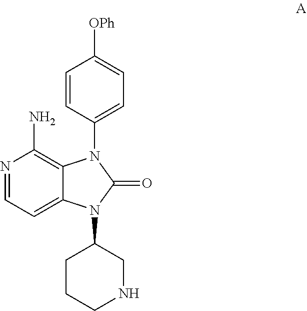 [SMILES: Nc1nccc2c1n(-c1ccc(Oc3ccccc3)cc1)c(=O)n2[C@@H]1CCCNC1]

or a salt thereof, with oxalic acid in a mixture of ethanol and water to form the compound of Formula (A-oxalate hydrate).

The present disclosure further relates to a compound of Formula (I):

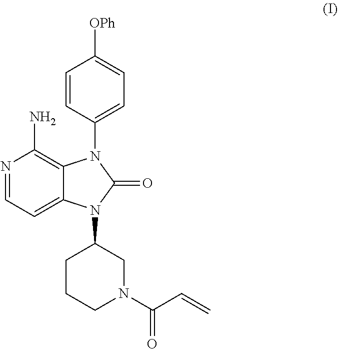 [SMILES: C=CC(=O)N1CCC[C@@H](n2c(=O)n(-c3ccc(Oc4ccccc4)cc3)c3c(N)nccc32)C1]

or a salt thereof, that comprises an impurity in an amount ranging from 0 to 1.4%.

The present disclosure furthermore relates to a compound of Formula (I):

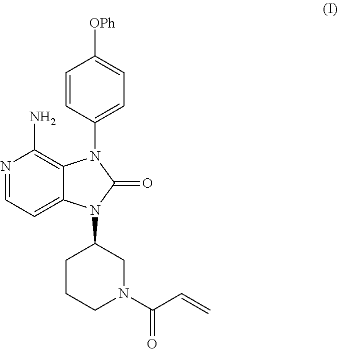 [SMILES: C=CC(=O)N1CCC[C@@H](n2c(=O)n(-c3ccc(Oc4ccccc4)cc3)c3c(N)nccc32)C1]

or a salt thereof, that is substantially free of an impurity compound of Formula (I)-Im:

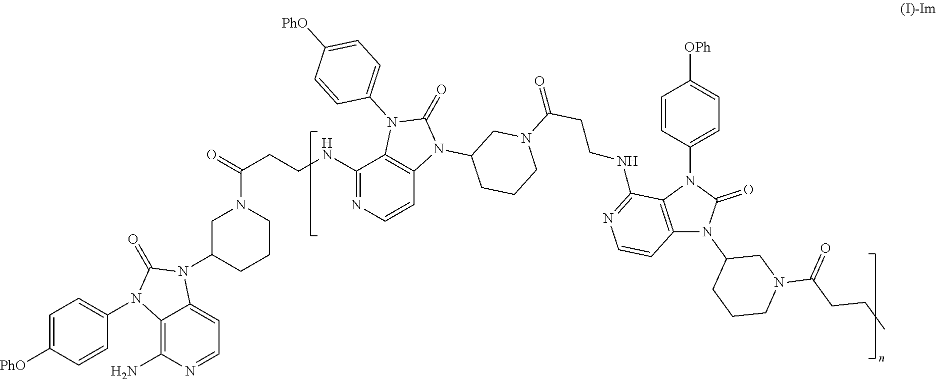

wherein n is equal to or greater than 1.

The present disclosure also relates to a compound of Formula (I)-Im:

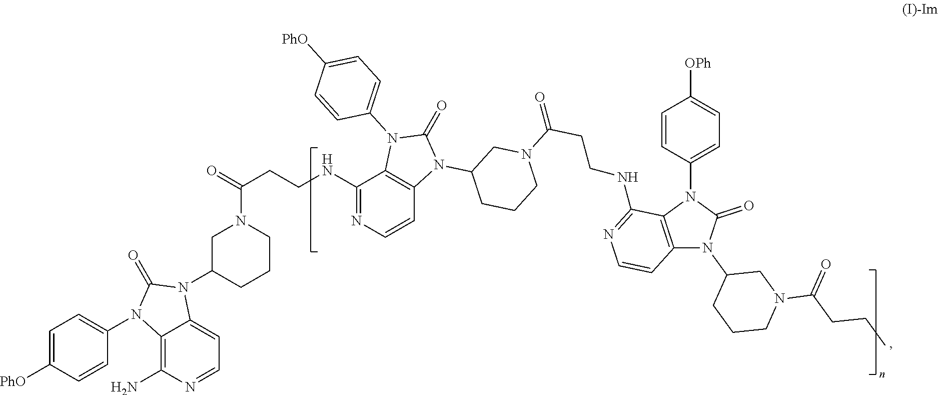

or a salt thereof.

The present disclosure further relates to a compound of Formula (A-oxalate hydrate):

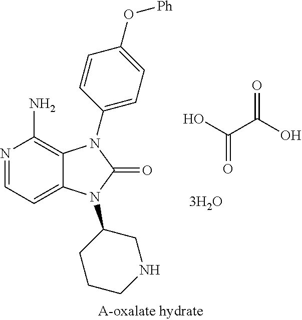 [SMILES: Nc1nccc2c1n(-c1ccc(Oc3ccccc3)cc1)c(=O)n2[C@@H]1CCCNC1 ; O=C(O)C(=O)O]

wherein the compound is about 90% pure.

The present disclosure furthermore relates to a crystalline form of a compound of Formula (A-oxalate hydrate):

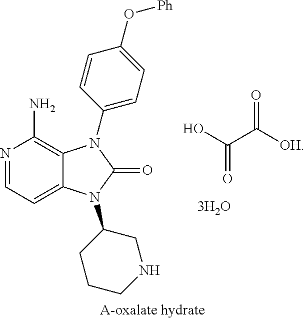 [SMILES: Nc1nccc2c1n(-c1ccc(Oc3ccccc3)cc1)c(=O)n2[C@@H]1CCCNC1 ; O=C(O)C(=O)O]

The present disclosure also relates to compositions comprising the compounds as disclosed herein.

### BRIEF DESCRIPTION OF THE DRAWINGS

FIG. 1 shows an ORTEP representative of the molecular structure of a compound of Formula A-oxalate hydrate in a crystal with thermal ellipsoids at 30% probability.

FIG. 2 shows a crystal structure of a compound of Formula A-oxalate hydrate with a representation of the molecular packing.

FIG. 3 shows a crystal structure of a compound of Formula A-oxalate hydrate with a focus on hydrogen bonds between the water molecules.

FIG. 4 shows hydrogen bonds and their bond lengths (Å) and angles (degrees °) in a crystal structure of a compound of Formula A-oxalate hydrate.

FIG. 5 shows crystal data and structure refinements of a crystal structure of a compound of Formula A-oxalate hydrate.

FIG. 6 shows atomic coordinates (×10⁴) and equivalent isotropic displacements parameters (Å2×103) in a crystal structure of a compound of Formula A-oxalate hydrate. U (eq) is defined as one third of the trace of the orthogonalized Uij tensor.

FIG. 7 shows bond lengths (A) in a crystal structure of a compound of Formula A-oxalate hydrate.

FIG. 8 shows bond angles (degrees °) in a crystal structure of a compound of Formula A-oxalate hydrate.

FIG. 9 shows hydrogen coordinates (×10⁴) and isotropic displacements parameters (Å²×10³) in a crystal structure of a compound of Formula A-oxalate hydrate.

FIG. 10 shows a molecular structure of a compound of Formula A-oxalate hydrate in a crystal showing the absolute configuration of the molecule.

FIG. 11 shows a simulated X-ray powder diffraction pattern from a crystal structure of a compound of Formula A-oxalate hydrate at copper wavelength (λ=1.54184 Å) and low temperature (T=100K).

FIG. 12A shows a ¹³C-CP-MAS NMR spectrogram of a compound of Formula (I) and FIG. 12B shows a ¹³C-CP-MAS NMR spectrogram of a compound of Formula (I)-Im.

FIG. 13 shows overlayed ¹³C-CP-MAS NMR spectrograms of a compound of Formula (I)-Im and a polymer obtained by polymerizing a compound of Formula (I) with azobisisobutyronitrile.

FIG. 14 shows chemical schemes for two different polymerization routes of a compound of Formula (I): FIG. 14A describes a radical polymerization of the vinyl groups of the compound of Formula (I) and FIG. 14B describes an aza-Michael addition between the amino and vinyl groups of the compound of Formula (I).

FIG. 15A shows a chemical scheme for acetylation of a compound of Formula (I) and FIG. 15B shows a 2D-¹H¹³C HSQC spectrogram of the acetylated reference polymer.

FIG. 16 shows ¹H-¹D-DOSY spectrograms of a sample spiked with 0.67% compound of Formula (I)-Im (top) and crude product comprising a compound of Formula (I) (bottom).

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings and Appendix, which are incorporated in and constitute a part of this specification, illustrate several embodiments and together with the description, serve to explain the principles described herein.

### DETAILED DESCRIPTION

### Definitions

Unless otherwise stated, the following terms used in the specification and claims are defined for the purposes of this disclosure and have the following meaning:

As used herein, “the BTK inhibitor,” “the BTK inhibitor compound,” “the compound of Formula (1),” “Compound 1,” and “the compound,” refers to (R)-1-(1-acryloylpiperidin-3-yl)-4-amino-3-(4-phenoxyphenyl)-1H-imidazo[4,5-c]pyridin-2(3H)-one having the following structure:

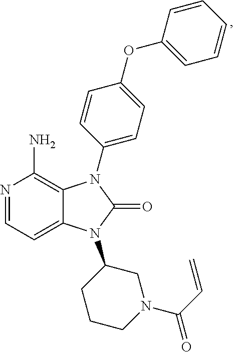 [SMILES: C=CC(=O)N1CCC[C@@H](n2c(=O)n(-c3ccc(Oc4ccccc4)cc3)c3c(N)nccc32)C1]

which is also known as 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

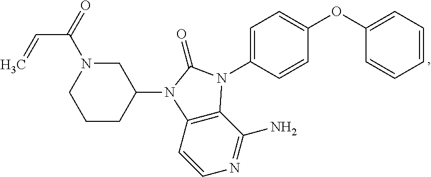 [SMILES: C=CC(=O)N1CCCC(n2c(=O)n(-c3ccc(Oc4ccccc4)cc3)c3c(N)nccc32)C1]

or a pharmaceutically acceptable salt thereof.

The present disclosure relates to a method of preparing a compound of Formula (I):

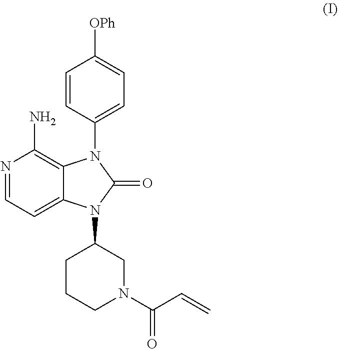 [SMILES: C=CC(=O)N1CCC[C@@H](n2c(=O)n(-c3ccc(Oc4ccccc4)cc3)c3c(N)nccc32)C1]

or a salt thereof, comprising:
reacting a compound of Formula (A-oxalate hydrate):

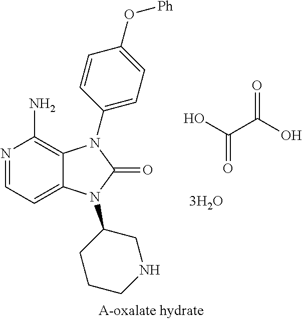 [SMILES: Nc1nccc2c1n(-c1ccc(Oc3ccccc3)cc1)c(=O)n2[C@@H]1CCCNC1 ; O=C(O)C(=O)O]

to form the compound of Formula (I), wherein the reaction comprises the following steps:
   (i) to a solution of the compound Formula (A-oxalate hydrate) adding 3-chloropropanoic acid; and
   (ii) to the mixture of (i), adding 1,8-diazabicyclo[5.4.0]undec-7-ene;
to prepare the compound of Formula (I).

In some embodiments, step (i) is performed in the presence of potassium carbonate, diisopropylethylamine, and propylphosphonic acid anhydride in dichloromethane.

In some embodiments, step (ii) is performed in dichloromethane.

In some embodiments, the method further comprises step (iii): washing the mixture of (ii) with hydrochloride.

In some embodiments, the method further comprises step (iv): dehydrating the organic layer obtained from (iii).

In some embodiments, the method further comprises step (v): adding seeds of compound (I)-HCl to the organic layer of (iv).

In some embodiments, the method further comprises step (vi): adding ethyl acetate to the mixture of (v) at a temperature ranging from 0 to 25° C.

In some embodiments, the method further comprises step (vii): filtering the solid from the mixture of (vi) and adding dichloromethane and sodium bicarbonate to the mixture.

In some embodiments, the method further comprises step (viii): filtering and concentrating the mixture of (vii).

In some embodiments, the compound of Formula (A-oxalate hydrate) is prepared by reacting a compound of Formula (A):

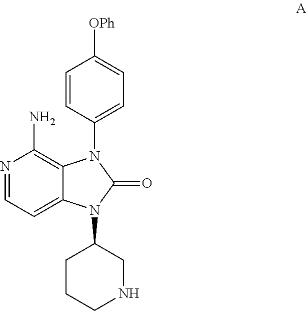 [SMILES: Nc1nccc2c1n(-c1ccc(Oc3ccccc3)cc1)c(=O)n2[C@@H]1CCCNC1]

or a salt thereof, with oxalic acid in a mixture of ethanol and water.

The present disclosure further relates to a method of preparing a compound of Formula (A-oxalate hydrate):

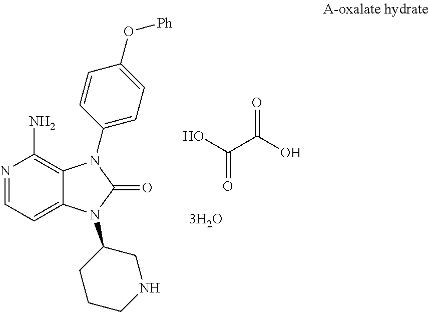 [SMILES: Nc1nccc2c1n(-c1ccc(Oc3ccccc3)cc1)c(=O)n2[C@@H]1CCCNC1 ; O=C(O)C(=O)O]

comprising:
reacting a compound of Formula (A):

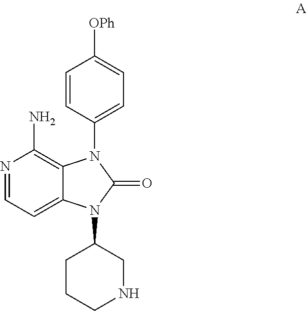 [SMILES: Nc1nccc2c1n(-c1ccc(Oc3ccccc3)cc1)c(=O)n2[C@@H]1CCCNC1]

or a salt thereof, with oxalic acid in a mixture of ethanol and water to form the compound of Formula (A-oxalate hydrate).

In some embodiments, the reaction comprises the addition of crystalline seeds of the compound of Formula (A-oxalate hydrate).

In some embodiments, the filtrate of (viii) comprises the compound of Formula (I), or a salt thereof, that is substantially free of an impurity compound of Formula (I)-Im:

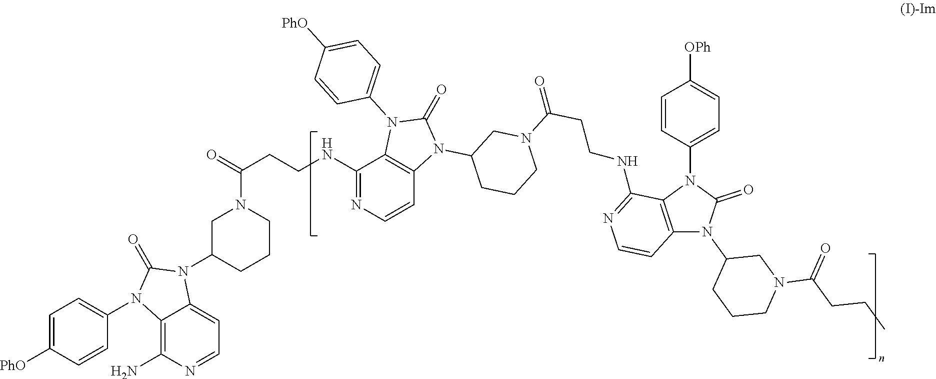

wherein n is equal to or greater than 1.

In some embodiments, the filtrate of (viii) yields a product that comprises the compound of Formula (I), or a salt thereof, and an impurity amount ranging from 0 to 1.40%.

In some embodiments, the impurity amount ranges from 0 to 0.900.

In some embodiments, the impurity amount ranges from 0 to 0.4%.

In some embodiments, the impurity is a compound of Formula (I)-Im:

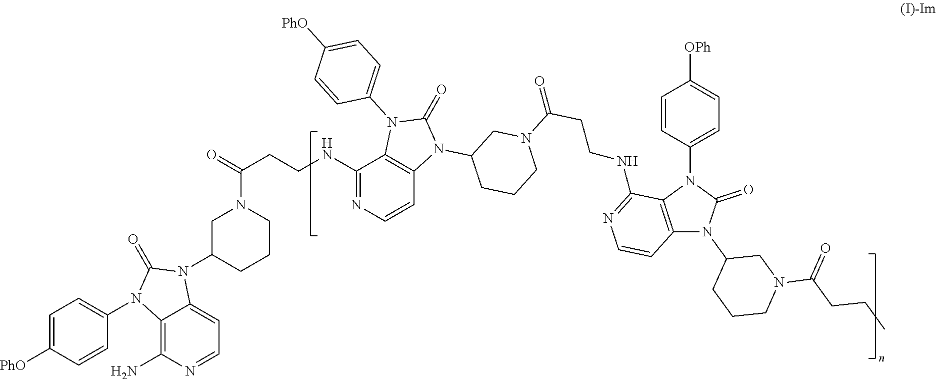

wherein n is equal to or greater than 1.

The present disclosure furthermore relates to a compound of Formula (I):

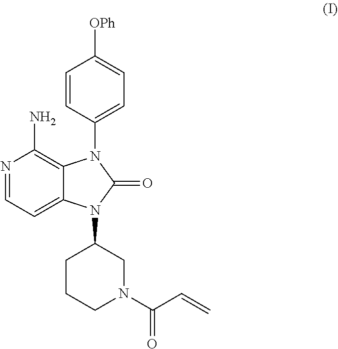 [SMILES: C=CC(=O)N1CCC[C@@H](n2c(=O)n(-c3ccc(Oc4ccccc4)cc3)c3c(N)nccc32)C1]

or a salt thereof, that comprises an impurity in an amount ranging from 0 to 1.4%.

In some embodiments, the impurity amount ranges from 0 to 0.9%.

In some embodiments, the impurity amount ranges from 0 to 0.4%.

The present disclosure also relates to a compound of Formula (I):

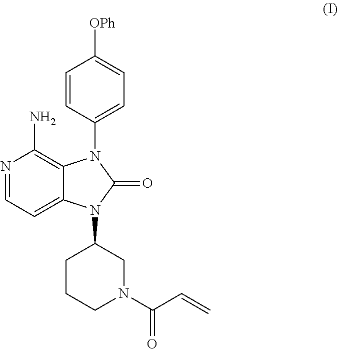 [SMILES: C=CC(=O)N1CCC[C@@H](n2c(=O)n(-c3ccc(Oc4ccccc4)cc3)c3c(N)nccc32)C1]

or a salt thereof, that is substantially free of an impurity compound of Formula (I)-Im:

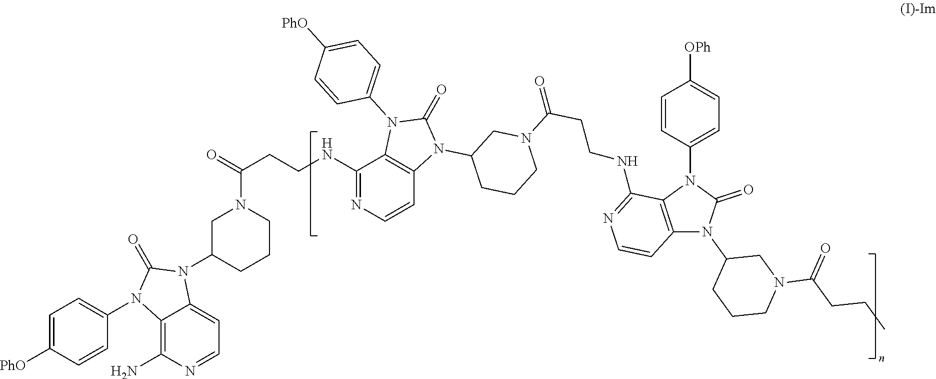

wherein n is equal to or greater than 1.

In some embodiments, the compound of Formula (I), or a salt thereof, comprises the impurity in an amount ranging from 0 to 1.4%.

In some embodiments, the compound of Formula (I), or a salt thereof, comprises the impurity in an amount ranging from 0 to 0.9%.

In some embodiments, the compound of Formula (I), or a salt thereof, comprises the impurity in an amount ranging from 0 to 0.4%.

In some embodiments, the impurity is a compound of Formula (I)-Im:

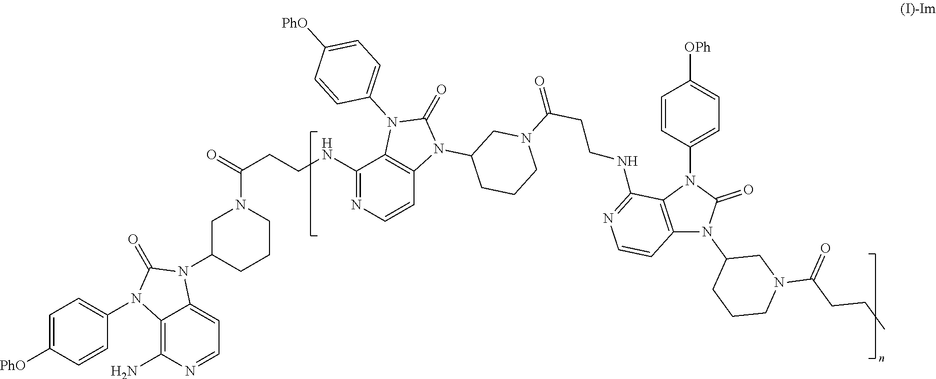

or a salt thereof, wherein n is equal to or greater than 1.

The present disclosure further relates to a composition comprising a compound of Formula (I) prepared by the methods described herein, wherein the composition is substantially free of an impurity compound of Formula (I)-Im:

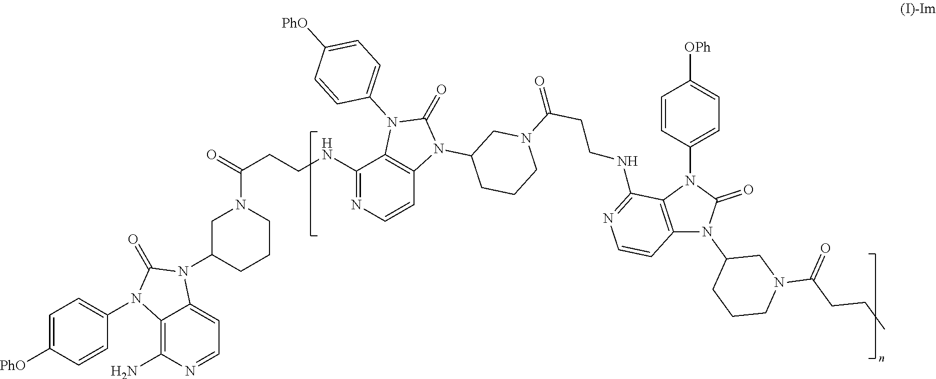

wherein n is equal to or greater than 1.

In some embodiments, the composition comprises at least 970% by weight or by HPLC analysis the compound of Formula (I).

In some embodiments, the composition comprises an impurity in an amount ranging from 0 to 1.4% by weight or by PLC analysis.

In some embodiments, the impurity amount ranges from 0 to 0.9% by weight or by HPLC analysis.

In some embodiments, the impurity amount ranges from 0 to 0.4% by weight or by HPLC analysis.

In some embodiments, the impurity is a compound of Formula (I)-Im:

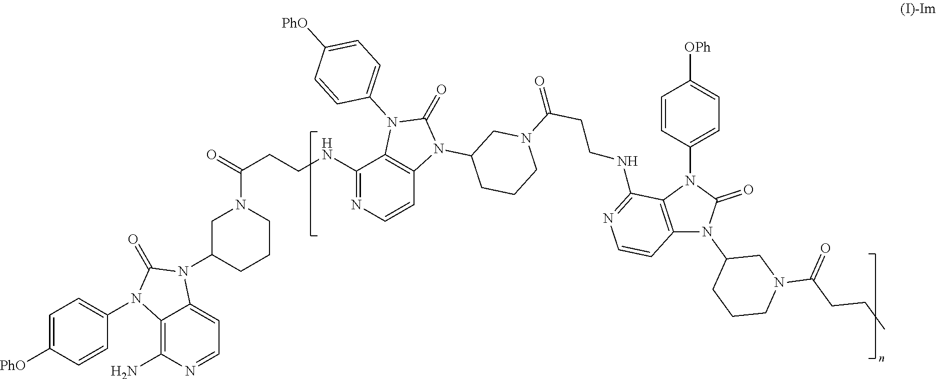

or a salt thereof, wherein n is greater than 1.

The present disclosure furthermore relates to a compound of Formula (I)-Im:

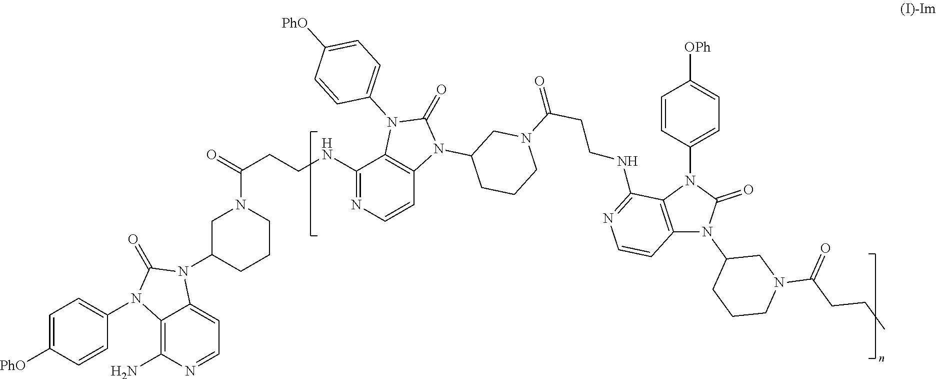

or a salt thereof, wherein n is equal to or greater than 1.

The present disclosure also relates to a compound of Formula (A-oxalate hydrate):

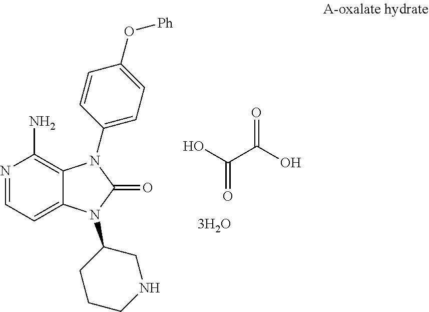 [SMILES: Nc1nccc2c1n(-c1ccc(Oc3ccccc3)cc1)c(=O)n2[C@@H]1CCCNC1 ; O=C(O)C(=O)O]

wherein the compound is about 90% pure.

In some embodiments, the compound is 95% pure.

In some embodiments, the compound is 98% pure.

The present disclosure further relates to a crystalline form of a compound of Formula (A-oxalate hydrate):

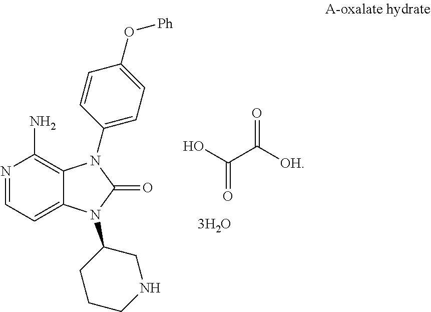 [SMILES: Nc1nccc2c1n(-c1ccc(Oc3ccccc3)cc1)c(=O)n2[C@@H]1CCCNC1 ; O=C(O)C(=O)O]

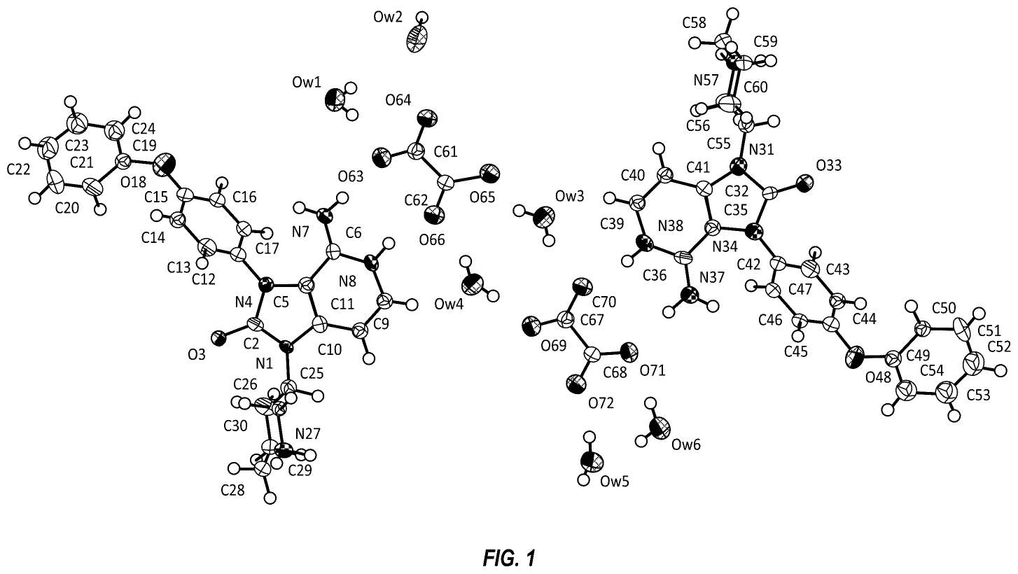

In some embodiments, the crystalline form has an ORTEP structure that is substantially in accordance with that shown in FIG. 1.

In some embodiments, the stoichiometry between the compound of Formula (A) and water in the crystalline form is 2:6.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following. “A; B; C; A and B; A and C; B and C; A, B and C”. An exception to this definition will occur only when a combination of elements, functions, steps or acts are in some way inherently mutually exclusive.

As used herein, “substantially free” means less than about 10% w/w, less than about 9% w/w, less than about 8% w/w, less than about 7% w/w, less than about 6% w/w, less than about 5% w/w, less than about 4.5% w/w, less than about 4% w/w, less than about 3.5% w/w, less than about 3% w/w, less than about 2.5% w/w, less than about 2% w/w, less than about 1.5% w/w, less than about 1.25% w/w, less than about 1% w/w, less than about 0.75% w/w, less than about 0.50% w/w, less than about 0.25% w/w, less than about 0.10% w/w, or less than about 0.05% w/w of impurities such as the compound of Formula (I-m). In some embodiments, “substantially free” means an undetectable amount of impurities such as the compound of Formula (I-m).

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In some embodiments, provided is a compound selected from the compounds in Table 1 or a salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described and within the scope of the claims.

TABLE 1

| Compound No. | Structure |
|---|---|
| A-oxalate hydrate | 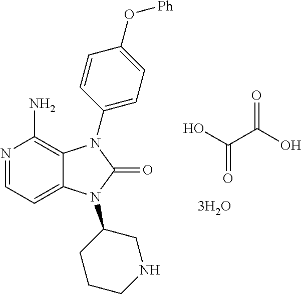 [SMILES: Nc1nccc2c1n(-c1ccc(Oc3ccccc3)cc1)c(=O)n2[C@@H]1CCCNC1 ; O=C(O)C(=O)O] |
| | A-oxalate hydrate |
| (I)-Im | 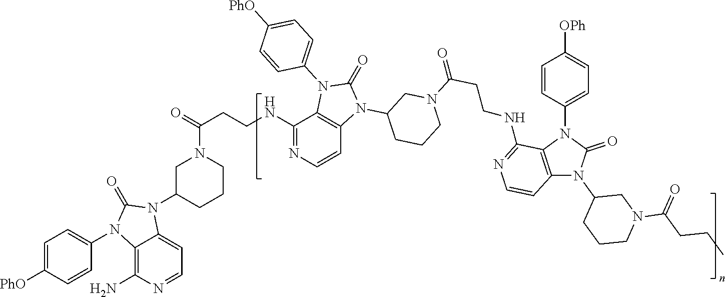 |
| | (I)-Im |

or a salt thereof.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

ACN or MeCN: acetonitrile
aq.: aqueous
DBU: 1,8-diazabicyclo[5.4.0]undec-7-ene
DCM: dichloromethane
DIPEA: N,N-diisopropylethylamine
EA, EtOAc, or AcOEt: ethyl acetate
equiv. or eq.: equivalents
EtOH: ethanol
h or hr: hour(s)
LCMS: liquid chromatography mass spectrometry
NMR: nuclear magnetic resonance spectroscopy
sat.: saturated
T3P: propylphosphonic acid anhydride
TFA: trifluoroacetic acid
V or vol.: volume per starting material (e.g., 10V solvent for 1 g of starting material=10 mL)

### EXAMPLE

### Example S1. Synthesis of (R)-1-(1-acryloylpiperidin-3-yl)-4-amino-3-(4-phenoxyphenyl)-1,3-dihydro-2H-imidazo[4,5-c]pyridin-2-one ((I))

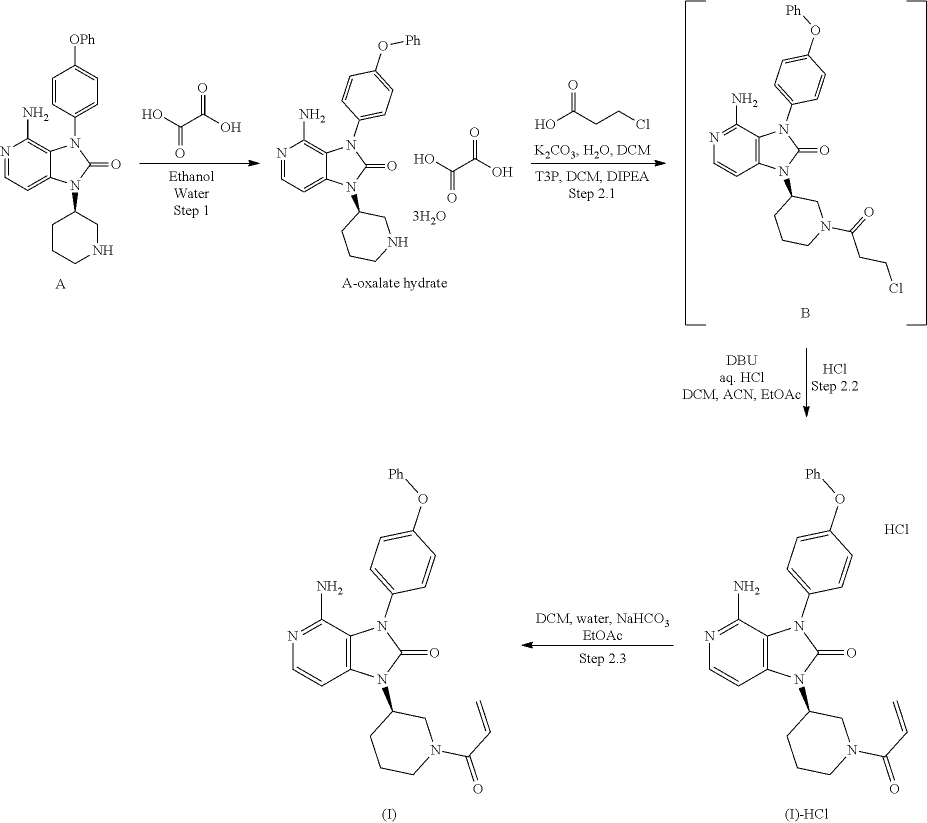

### Example S1.1. (R)-4-amino-3-(4-phenoxyphenyl)-1-(piperidin-3-yl)-1,3-dihydro-2H-imidazo[4,5-c]pyridin-2-one oxalate trihydrate (A-oxalate hydrate)

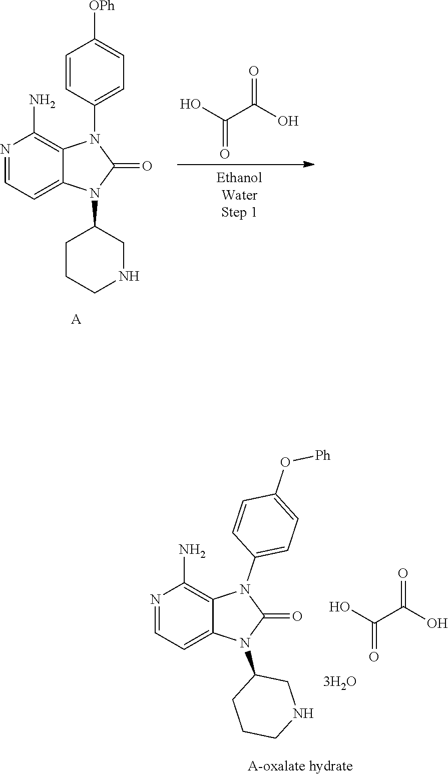

To compound A dissolved in 5.3V EtOH was added a solution of oxalic acid (1.0 eq) in 2.7V water at 50° C. The solution was cooled to 45° C., to which a seed of A-oxalate hydrate (0.01 kg/kg, synthesis procedure described below) was added. The suspension was stirred 2 h, cooled to 30° C., and stirred for another 5 h at 30° C. The mixture was then cooled to 0° C., and the solid was filtered off. A-oxalate hydrate was obtained as a solid (70-77% purity, 88% yield).

The seed of A-oxalate hydrate used in the above procedure was prepared by dissolving 10 g of compound A and oxalic acid in 50 mL of water at 41° C., and adding to this mixture 100 mL of absolute ethanol over 90 min under stirring. Slow nucleation after 2.5 h at 43° C. was observed. The suspension was cooled down to 20° C. and the product was collected.

Analysis of the crystal structure of A-oxalate hydrate indicated the crystal comprises two molecules of compound A, two molecules of the counter ion oxalic acid, and six molecules of water. Details of the studies are described as follows.

A single crystal was isolated and selected by observation under a binocular microscope mounted on the goniometric head of a Rigaku Oxford CrysAlisPro diffractometer. Intensities were collected with the diffractometer at low temperature (T=100 K), with the use of a micro focused Cu Kα radiation wavelength (λ=1.54184 Å). Systematic investigation of the diffraction nodes indicated that the crystal belongs to the triclinic system, with a primitive Bravais lattice. The unit cell parameters are given below:

| a (Å) = | b (Å) = | c (Å) = | α (°) = | β (°) = | γ (°) = |
|---|---|---|---|---|---|
| 9.67 | 11.68 | 11.75 | 83.58 | 80.81 | 79.08 |

In view of the number of atoms in the compound A molecule and of the unit cell volume, it was concluded that the unit cell must contain 2 molecular entities having the formula C₂₅H₃₀N₅O₉, which is equivalent to a calculated density of 1.412. The number of reflections collected was 13201, of which 7056 were unique. Based on the statistical distribution of the intensities, a non-centrosymmetric structure was deduced.

The structure was solved by direct methods using the XT dual-space module of SHELX and was refined on F² by full least squares methods with SHELXTL. The molecular structure was well found. All non-hydrogen atoms were refined with anisotropic displacement parameters, and a riding model was used for hydrogen atoms. Final agreement values were R1=0.0473 (observed reflections) and wR2=0.1288 (all data) for 7056 reflections and 736 parameters, with a goodness of fit of 1.054.

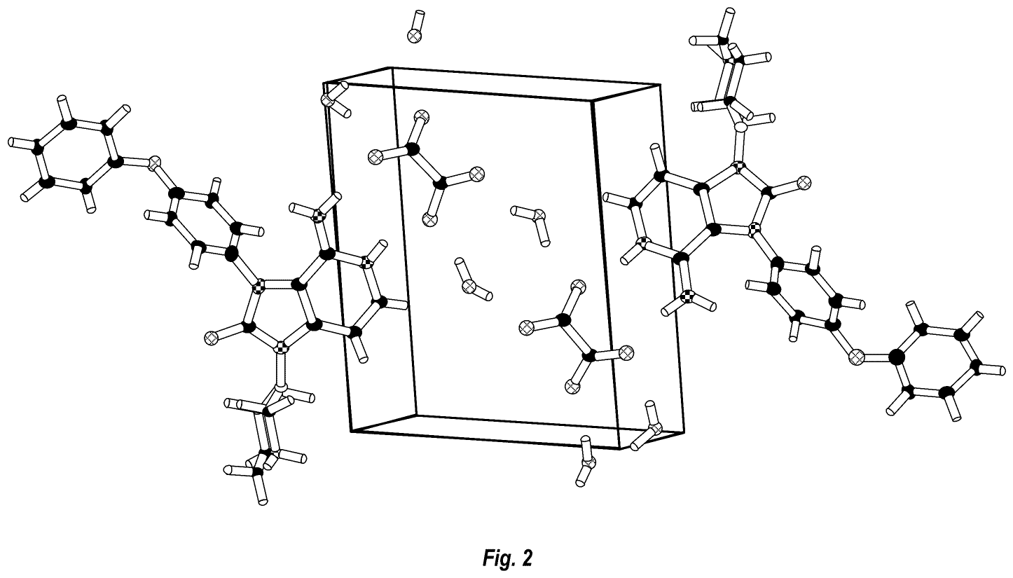

The compound was shown to crystallize in the space group P1. The asymmetric unit of the crystal was made up of two molecules of compound A together with two molecules of counter ion oxalic acid and six molecules of water (FIG. 1 and FIG. 2). Therefore, 2 formulae were present in the unit cell. The crystal structure corresponded to a trihydrate, and the asymmetric unit therefore contained: [2(C₂₃H₂₅N₅O₂), 2(C₂O₄), 6(H₂O)]. Examination of the molecular structure confirmed that all bond angles and lengths stood in the standard range values. No disorder appeared to be present in the crystal.

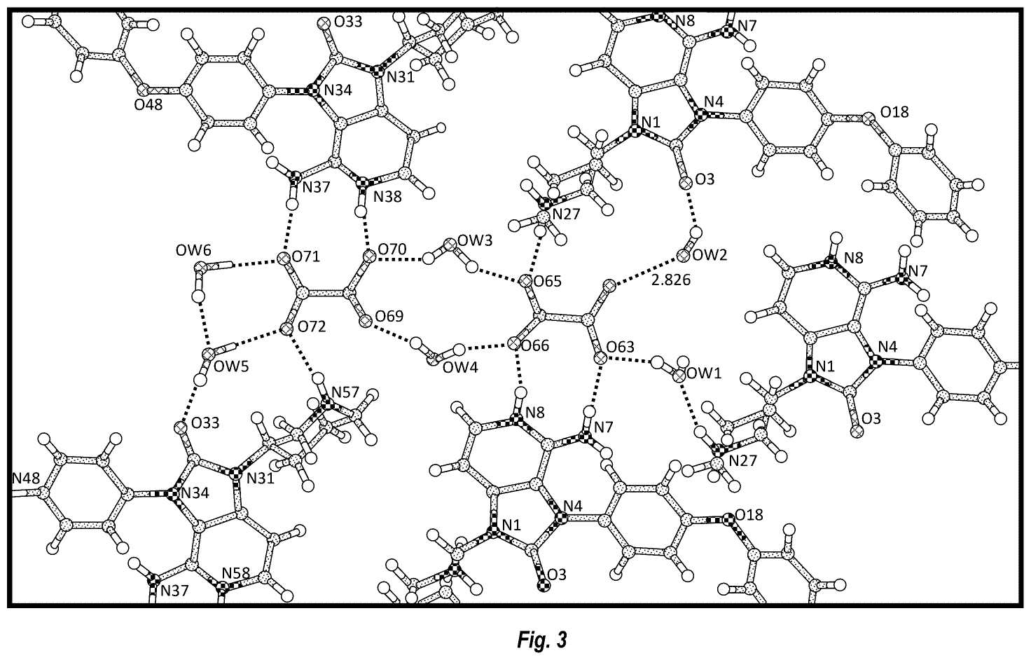

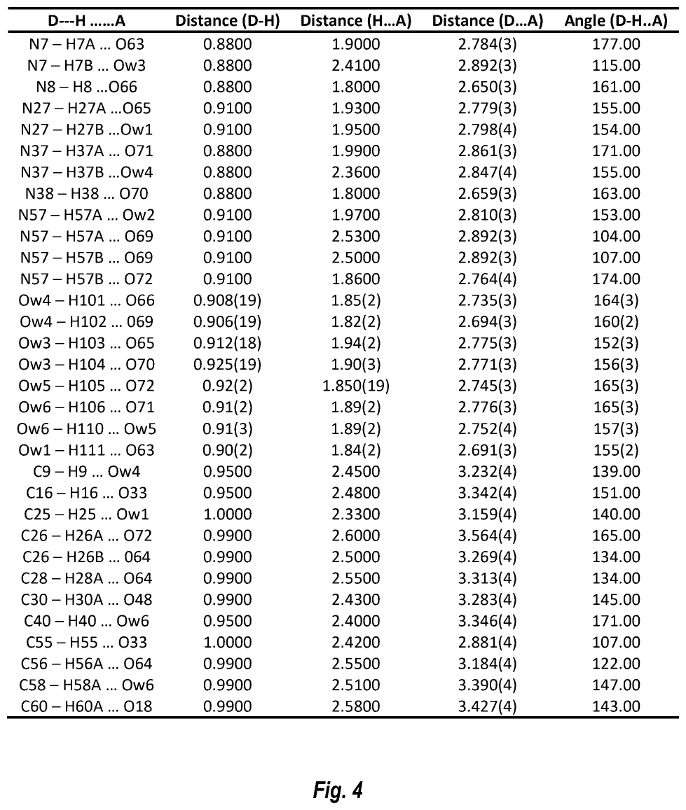

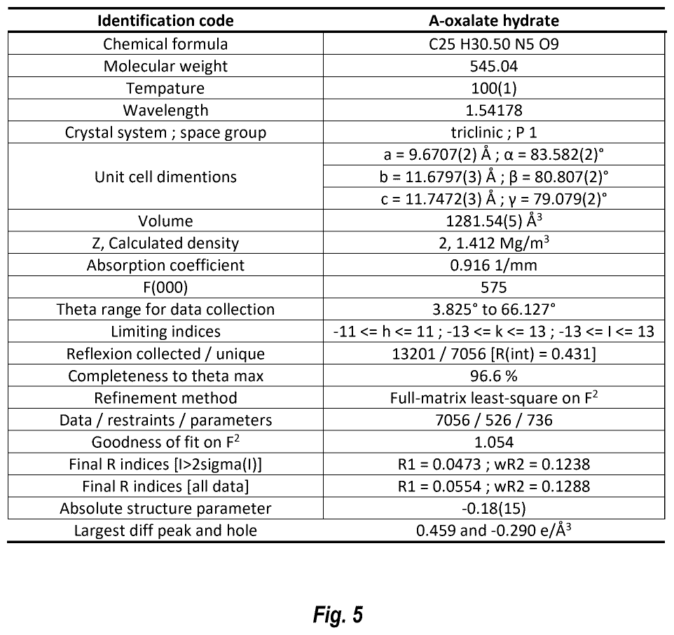

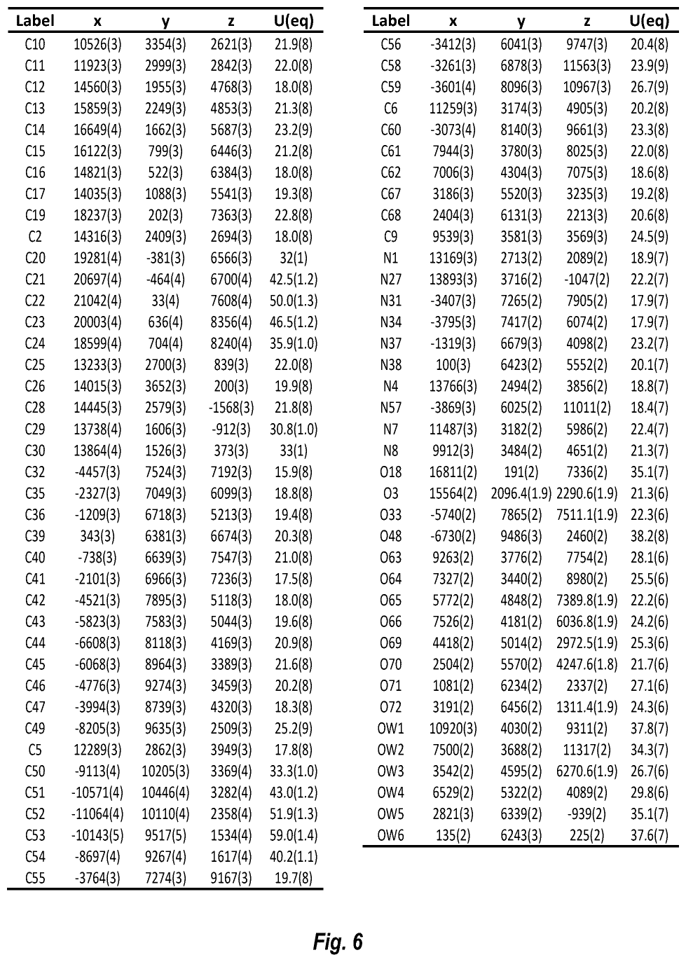

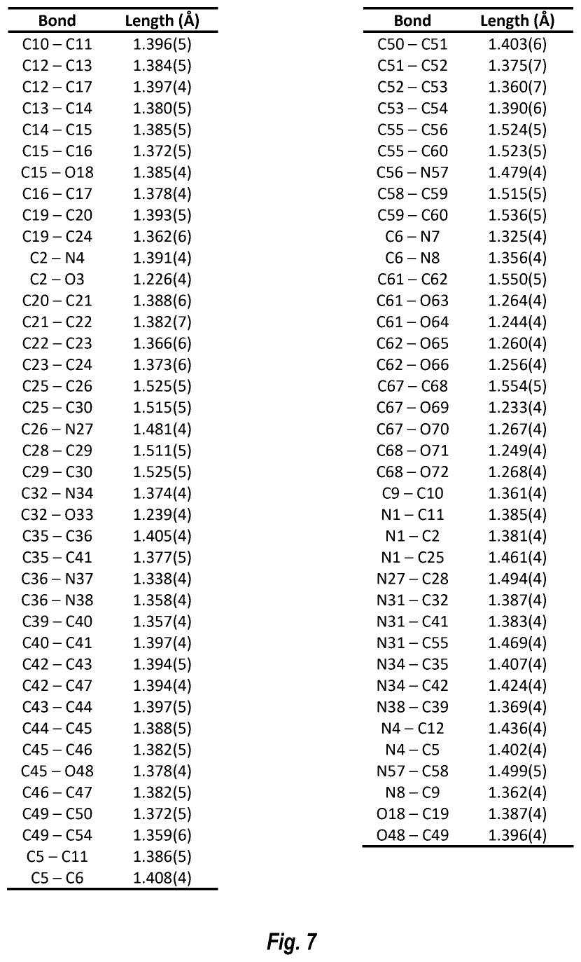

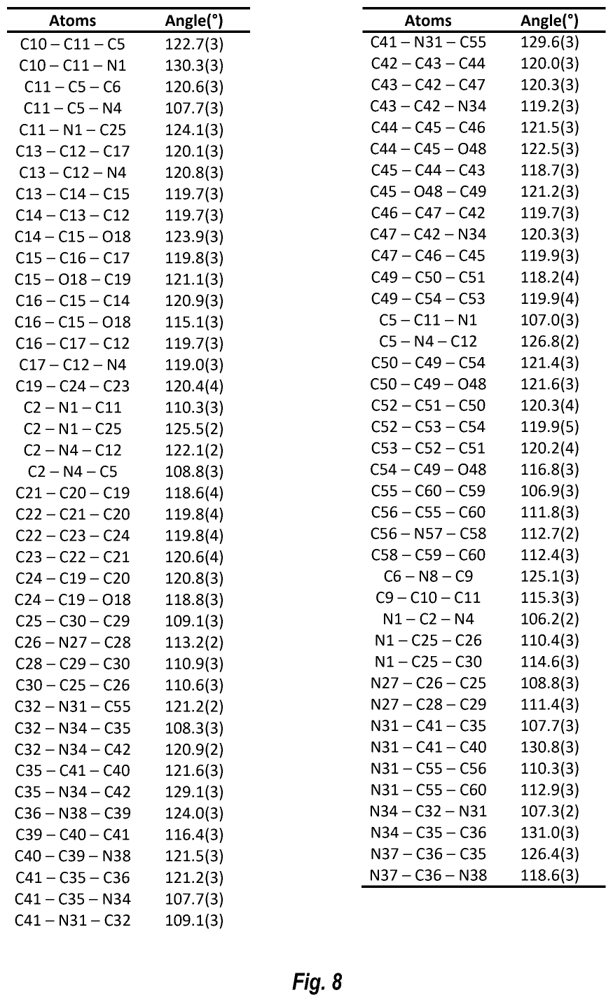

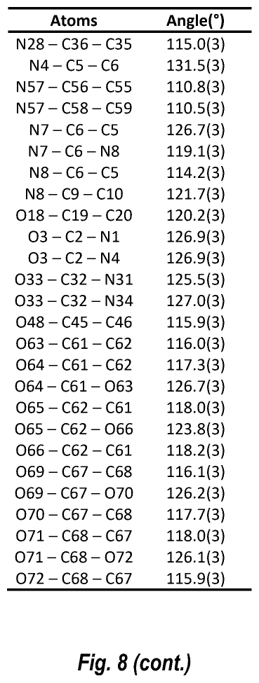

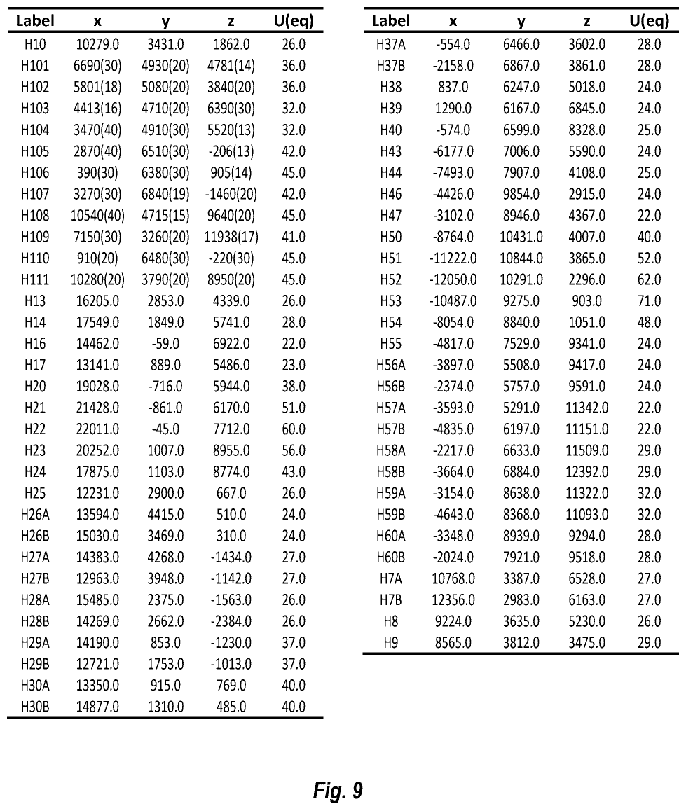

The hydrogen bonds were as shown in FIG. 3. Other non-covalent interactions were also present in the structure as shown in FIG. 4. The crystal data, X-rays experimental parameters, and structure refinements were as shown in FIG. 5. The positional parameters for all independent non-hydrogen atoms together with their equivalent isotropic displacement parameters were as shown in FIG. 6. Bond lengths and angles were as shown in FIG. 7 and FIG. 8. The position of the hydrogens were as shown in FIG. 9, and all the hydrogen bonds were as shown in FIG. 4. The PLATON program was used to generate crystal structures and perform structure validation.

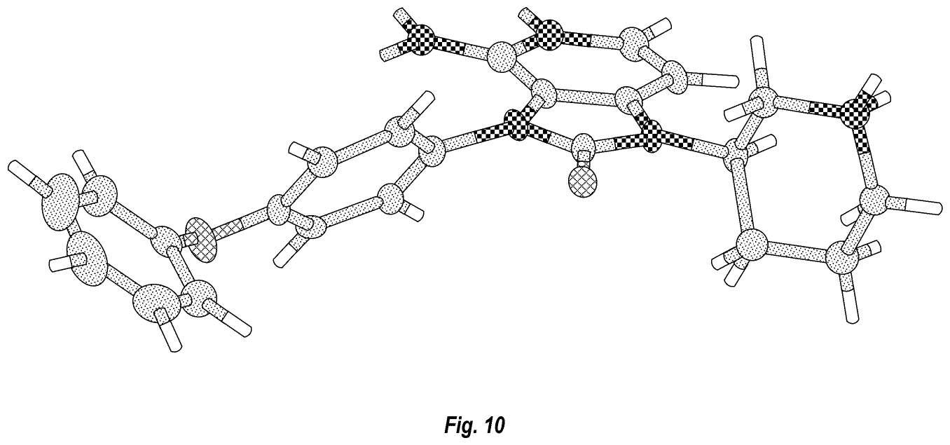

The compound A molecule did not contain a heavy atom that could easily allow the absolute configuration to be determined. Nevertheless, thanks to the fraction of oxygen in the molecule with respect to carbon, it was possible to elucidate the absolute configuration making use of a high-resolution data collection (performed at low temperature). The Flack x parameter was calculated based on the anomalous scattering method, which gave the absolute structure. The absolute configuration was as shown in FIG. 10.

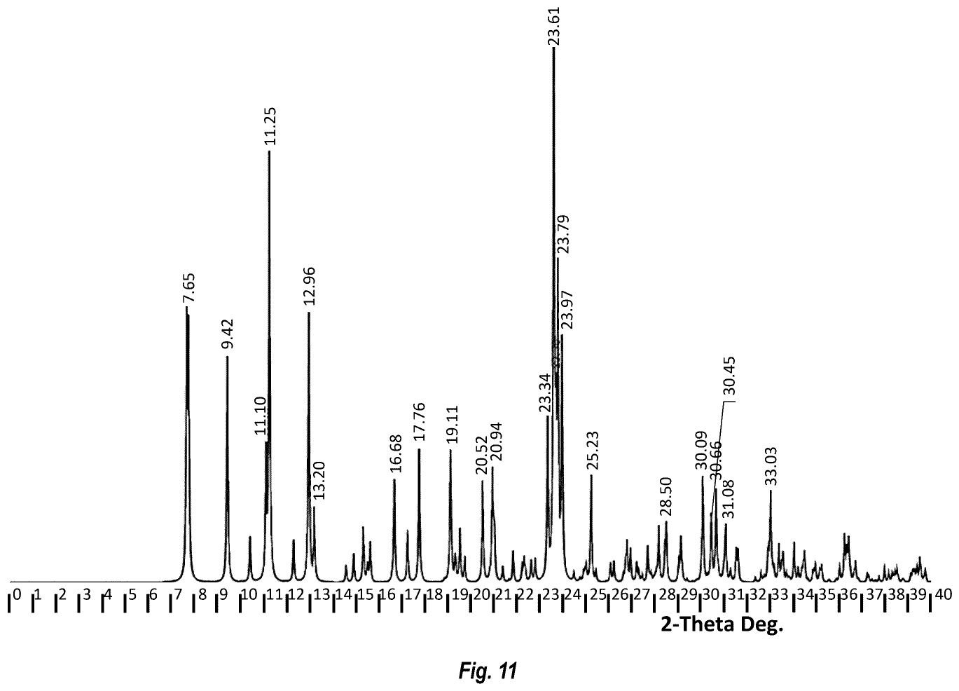

A simulated diffraction pattern (FIG. 11) was produced from the experimentally determined crystal structure of A-oxalate hydrate.

### Example S1.2. I-1-(1-acryloylpiperidin-3-yl)-4-amino-3-(4-phenoxyphenyl)-1,3-dihydro-2H-imidazo[4,5-c]pyridine-2-one ((I))

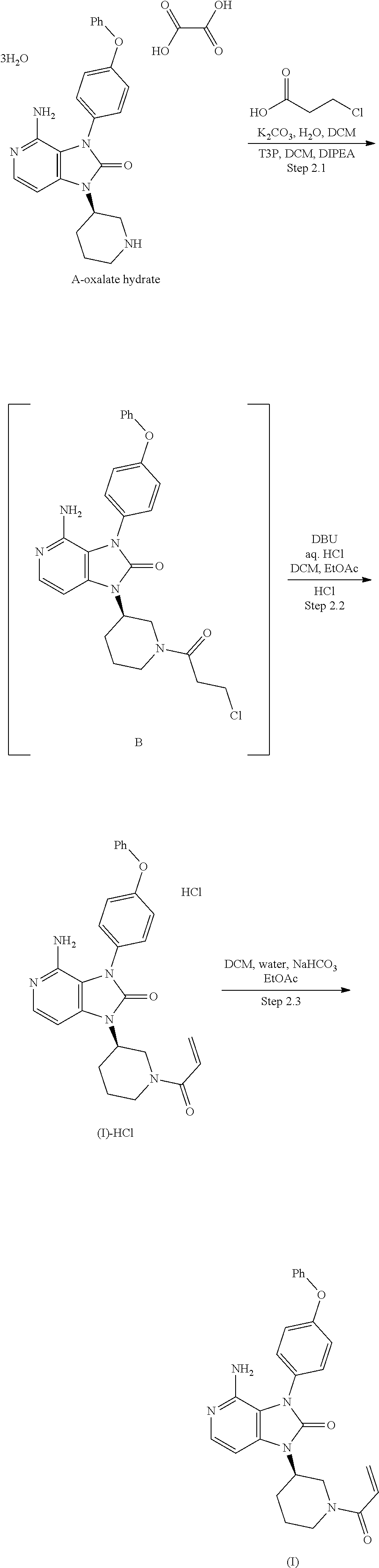

Purified water (7.5 vol.) and K₂CO₃ (at least 3.0 eq.) was added to compound A-oxalate hydrate (corresponding to 1 eq. compound A) in DCM (12 vol.) at 20° C., and the reaction mixture was stirred for at least 2 h. The reaction mixture was then allowed to settle and separate. The organic layers were collected and washed 1-2 times with water (7.5 vol.) to afford A-oxalate hydrate in DCM solution. The solution was concentrated to 12 vol. and mixed with DIPEA (3.5 eq.) at 20° C. Next, a solution of 3-chloropropanoic acid (1.02 eq.) in DCM (2.3 vol.) and T3P (50% DCM solution, 1.1 eq.) was added at 20° C. Compound B was formed in situ. Next, DBU (4 eq.) was added to the reaction mixture at 30° C. over at least 30 min. and the resulting mixture was kept at 30° C. for at least 4 hr. The organic layer was washed at least 6 times with HCl (1 N, 10 vol.) at 20° C. Next, the organic layer was dehydrated and then concentrated to 3 vol. at 8° C. The temperature of the organic layer was then adjusted to 15° C. Compound (I)-HCl seeds (0.01 kg/kg) were added to the organic layer at 15° C., and the mixture was stirred for 2.5 h. Ethyl acetate (1.6 vol.) was then added at 15° C., and the mixture was stirred for 3.5 h. Then, the mixture was then cooled to 0° C. and stirred for 4 h. A filter-dryer was charged with the resulting suspension, and the solid was filtered and washed with ACN (5.4 vol.), which yielded (I)-HCl (70% yield).

To (I)-HCl was added DCM (12 vol.) at 10° C., and this solution was washed with water (10 vol.). The aqueous phase was extracted with DCM (2 vol.), which was combined with the main organic phase. Base release was performed twice with a saturated aq. NaHCO₃ solution (7.6 vol.), and the organic phase was washed with water four times. The resulting solution was treated with charcoal, filtered, and concentrated at 15° C. to 2.64 vol. EtOAc was added in two parts up to a total of 4.84 vol. at 40° C., followed by a solvent exchange to DCM (4.84 vol.) at 40° C. This solution was seeded at 40° C. with 0.018 kg/kg of the compound of Formula (I), prepared according to the procedure described in WO2023/244587. The suspension was wet milled at 40° C. and was then cooled to 0° C. and stirred for at least 2 h. The solid was filtered, washed twice with EtOAc (1.76 vol. each), and dried in vacuo at a temperature up to 50° C. to yield the compound of Formula (I).

It should be noted that the steps above may be performed at a temperature at or below 40° C. (such as, for example, at 20 or 30° C.) in dichloromethane. In some embodiments, it has been found that by keeping this lower temperature, the polymerization reaction forming an impurity will be reduced such that the impurity may have a concentration of less than 600 ppm, which is the purity requirement set by the United States Food and Drug Administration (US FDA). Thus, by controlling the solvent and the temperature, the material produced may meet the purity requirements needed for a commercial batch of the compound of Formula (I).

The following elemental analysis was performed by Galbraith Laboratories to characterize the final compound: Carbon, Hydrogen, and Nitrogen Determination using the PerkinElmer2400 Series II CHNS/O Analyzer and determination of Total Halogens or Total Halides by Potentiometric Titration.

NMR and LCMS data were consistent with compound (I).

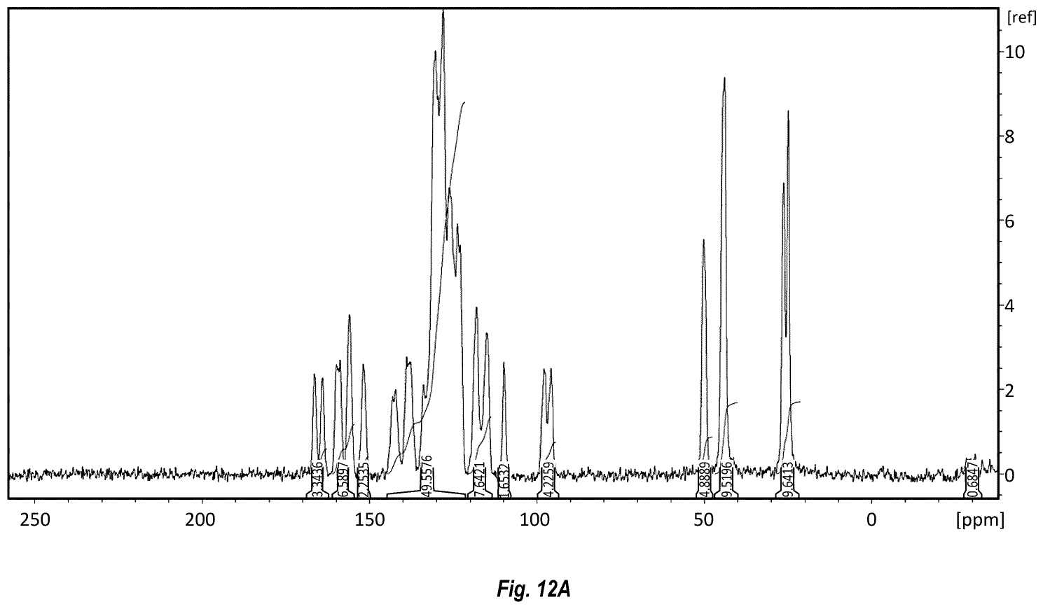

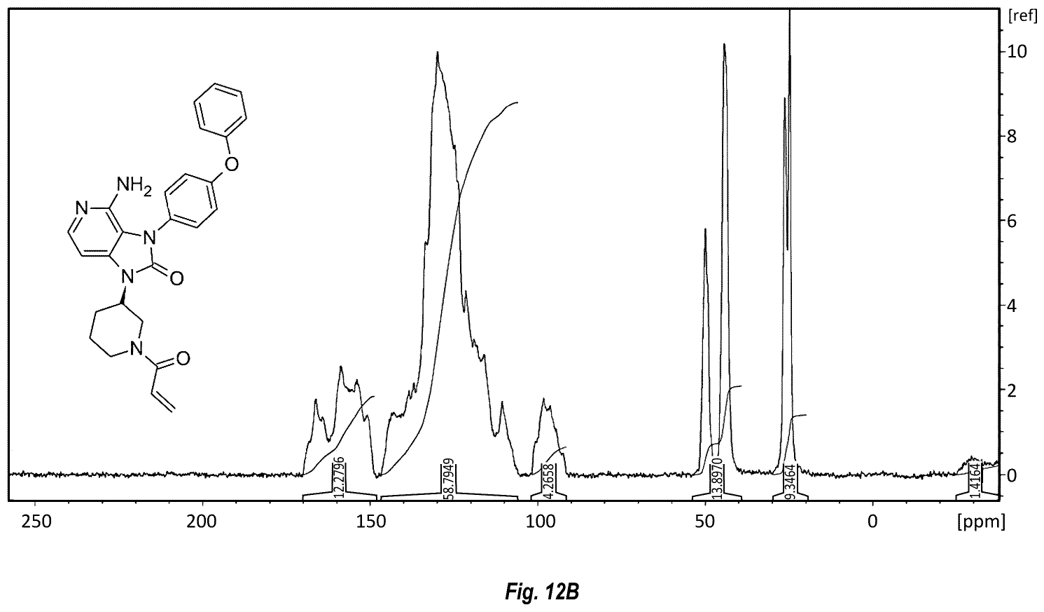

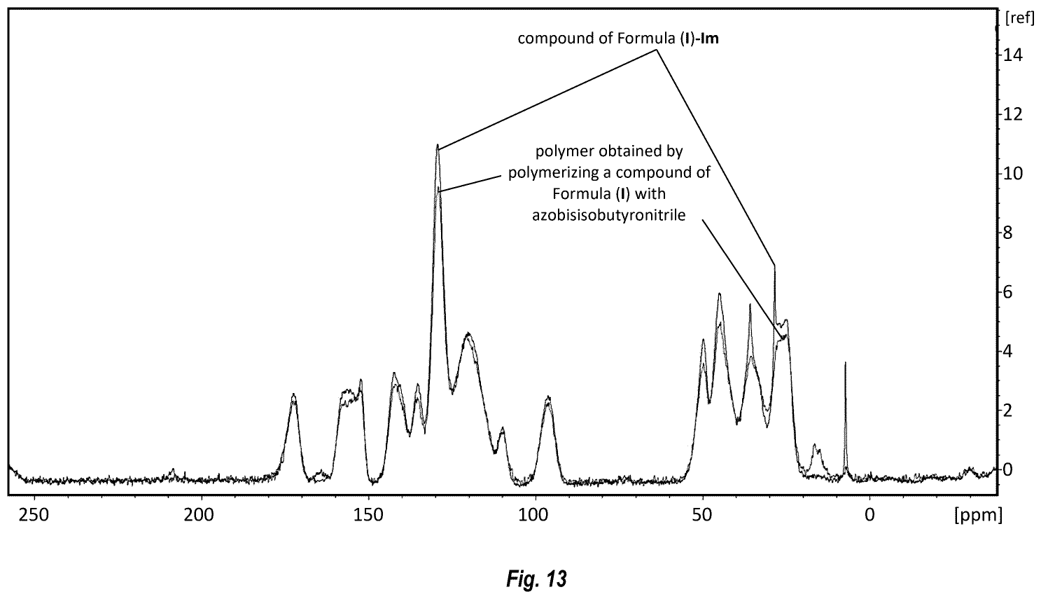

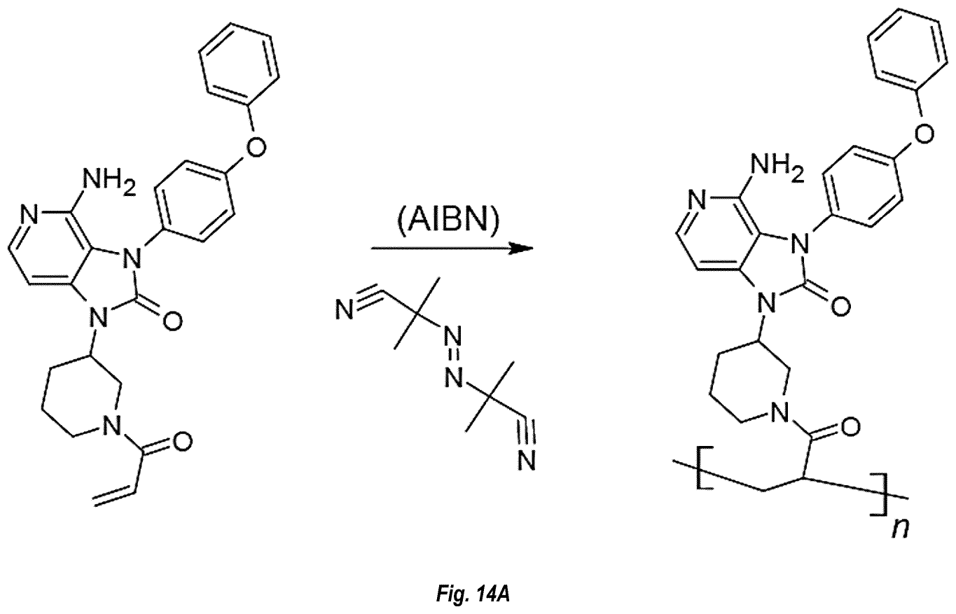

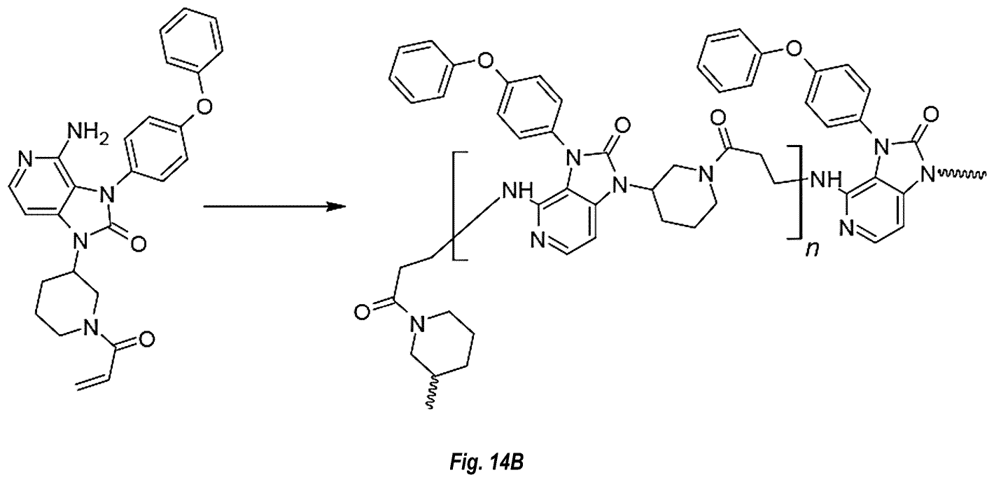

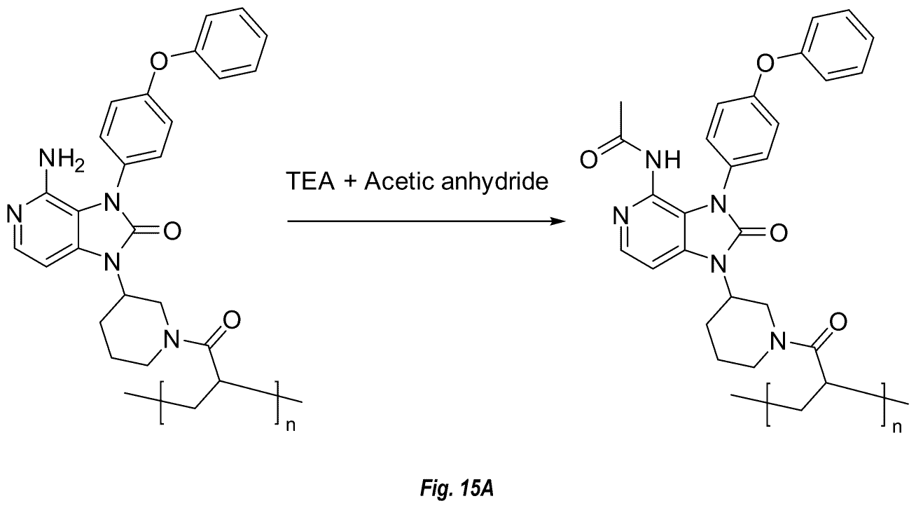

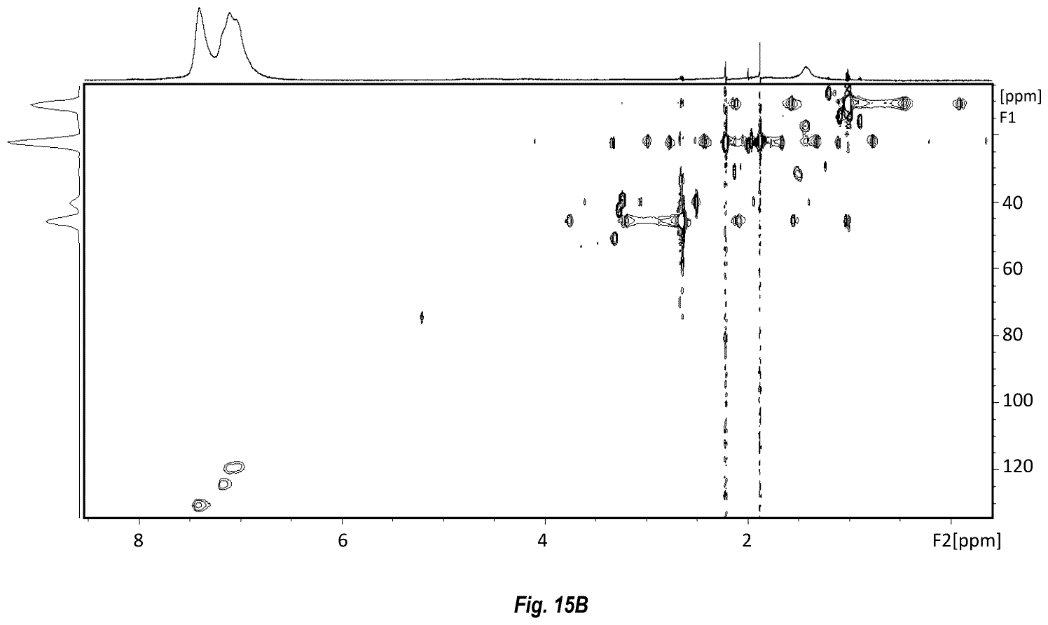

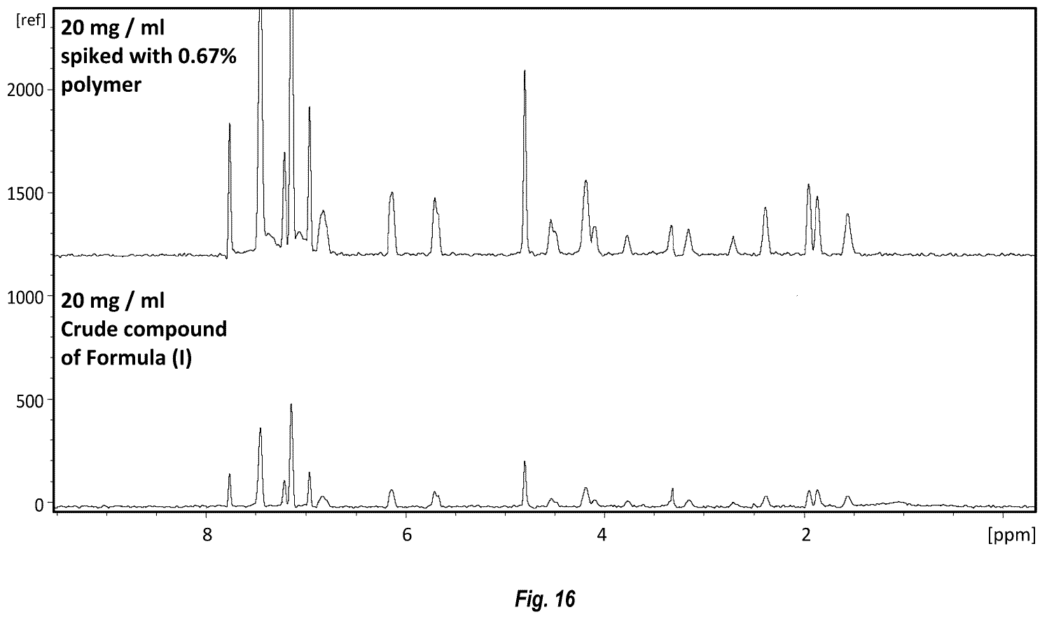

NMR analysis of the final compound confirmed the compound of Formula (I) was obtained with a >97% purity. Multiple types of impurities were identified, wherein the total impurity content was lower than 3%, and the content of each of the impurities were below 1.4%. One impurity in particular, the compound of Formula (I)-Im, which was isolated from the final product via dissolution in acetonitrile and filtration, was characterized in detail. ¹³C-CP-MAS NMR characterization of the compound of Formula (I)-Im (FIG. 12B) showed correlated signals with the compound of Formula (I) but displayed broader resonance linewidths (FIG. 12A). These results indicated the impurity is a polymerized form of the compound of Formula (I). For verification purposes, the compound of Formula (I) was polymerized with azobisisobutyronitrile to yield a “reference polymer,” which was analyzed by ¹³C-CP-MAS NMR and compared to the compound of Formula (I)-Im (FIG. 13). As expected, the compound of Formula (I)-Im and the reference polymer showed matching signals and resonance linewidths, indicating the compound of Formula (I)-Im is indeed a polymerized form of the compound of Formula (I). To confirm whether the compound of Formula (I)-Im is formed by radical polymerization of the vinyl groups (FIG. 14A) or aza-Michael addition between the amino and vinyl groups of the compound of Formula (I) (FIG. 14B), the compound of Formula (I)-Im was subjected to acetylation conditions and analyzed by 2D-¹H¹³C HSQC. The reference polymer was also subjected to the same conditions and analyzed. The compound of Formula (I)-Im did not show signs of substitution, indicating the amine groups were unavailable, unlike the reference polymer that showed clear signs of acetylation of the amine group (FIG. 15A and FIG. 15B). Overall, the results indicated that the compound of Formula (I)-Im resulted from the polymerization of the compound of Formula (I) via Michael addition. ¹H-¹D-DOSY studies confirmed the content of the compound of Formula (I)-Im was below 0.67%, which was the limit of detection of the NMR (FIG. 16).

As noted above, step 2.2 in Example S1.2 was conducted in DCM to maximize the solubilization of the compounds in the reaction mixture. Once the reaction was complete and the DCM solution containing the crude (I)-HCl was concentrated, insoluble species could be observed in the solution. These insoluble impurities could remain in the crude (I)-HCl powder obtained after the crystallization step, and when these batches of crude (I)-HCl powder comprising the insoluble impurities were processed for the next step (step 2.3 in Example S1.2), insoluble species could be observed again in the DCM and aqueous phase. Based on these observations, it was concluded that these insoluble species were related to the crude (I)-HCl. These polymer impurities could also appear during the solvent change from DCM to ethyl acetate in step 2.3 in Example S1.2. Although they could be isolated to yield the pure compound of Formula (I), these insoluble species could cause inefficiencies in the synthesis and purification process.

It was discovered that performing step 2.2 in Example S1.2 in DCM at temperatures below 20° C. results in reduced polymer impurities, and the amount of impurity found in the crude (I)-HCl powder obtained after the crystallization step was nearly negligible. The polymer impurities also remained negligible after the crude (I)-HCl powder was dissolved in DCM and water in step 2.3 in Example S1.2. As a result, the filtration and separation of the two phases described above in step 2.3 in Example S1.2 could be performed under 1 h, whereas the same process in the presence of insoluble impurities could take up to several hours.

Therefore, the process in step 2.2 in Example S1.2 was modified to concentrate the DCM solution at a temperature below 20° C., which reduced the kinetics of the insoluble impurity formation, as compared to the prior disclosure (WO2023/244587), wherein the temperature was chosen to be 60° C. Crystallization conditions, in particular, the seeding temperature and isothermal stage, were also modified to enable feasible isolation of the product in an industrial reactor.

Further, in step 2.3 in Example S1.2, to eliminate the small quantities of residual insoluble material during the liquid-liquid extractions, after carrying out filtration, the DCM solution was sent to an additional clean reactor. Then, the reactor in which this first liquid-liquid extraction was carried out was cleaned to avoid recontamination of the DCM solution with these residual insoluble impurities, which was observed to stick to the wall of the reactor.

Further chemical stability studies varying operating conditions of the process (temperature, pure DCM solvent or water, binary DCM/water, dilution of the medium) showed that the (I)-HCl molecule forms polymers in DCM and that the kinetics of this reaction are rapid at high temperature, for example, near 60° C., which was the initial setpoint for the process in the prior disclosure (WO2023/244587). It was observed that the polymer formation is avoided when ethyl acetate is added to DCM. In other words, while DCM was chosen to improve solubility of the materials, at a sufficiently high temperature the use of DCM could lead to formation of the polymer impurities. However, we note that using DCM at a sufficiently low temperature, but high enough to avoid spontaneous crystallization, prevented impurity formation, which is an innovative aspect of the present disclosure. For instance, the distillation conditions were modified to concentrate the DCM solution comprising (I)-HCl to a working concentration at a temperature below 20° C. This adjustment made it possible to limit the formation of polymer impurities because of the slower kinetics at lower temperature.

The temperature of the solution was then raised stepwise during the controlled addition of ethyl acetate in step 2.3 in Example S1.2. Thus, when the double jacket temperature reached 40-45° C. in the reactor, there was already sufficient amount of ethyl acetate in the solution and the formation of polymers did not take place. The industrial advantage of initially concentrating the DCM solution to a much lower volume was that it could ultimately reduce the length of time wherein the mixture containing the product has to be subjected to heat, as a lower solvent volume could reduce the duration of distillation, which could ultimately reduce the polymer impurity formation. Another advantage was that it could reduce the processing time because there is less ethyl acetate to distill and therefore the process time is reduced, which also contributes to limiting the heating times of the DCM solution containing ethyl acetate. This helped control polymer formation.

Laboratory tests confirmed that the formation of polymer impurities occurred on the hot walls of the reactor while the DCM solution was under stirring and in contact with the heat transmitting wall.

The crystallization process in Example S1.2 was also adjusted in order to reduce and control the formation of polymers. It would be preferable to work at low temperatures, close to 20° C., when performing the solvent exchange and crystallization. However, this was not possible since when crystallized below 35° C., (I)-HCl would trap DCM and it would no longer be able to be dried sufficiently enough to comply with the industrial specification that require residual DCM below 600 ppm in the final powder. Therefore, the temperature during the entire solvent change and crystallization process was carried out at a temperature between 35-45° C.: above 35° C. to avoid the trapping of DCM in the solid, and below 45° C. to avoid the formation of polymers.

Although the present disclosure has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

Embodiment 1. A method of preparing a compound of Formula (I):

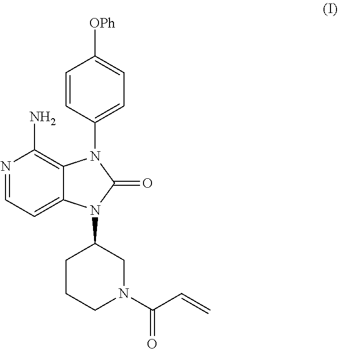 [SMILES: C=CC(=O)N1CCC[C@@H](n2c(=O)n(-c3ccc(Oc4ccccc4)cc3)c3c(N)nccc32)C1]

or a salt thereof, comprising:
reacting a compound of Formula (A-oxalate hydrate):

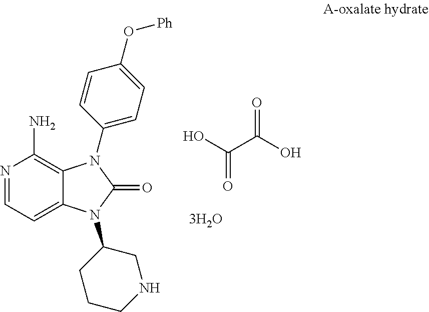 [SMILES: Nc1nccc2c1n(-c1ccc(Oc3ccccc3)cc1)c(=O)n2[C@@H]1CCCNC1 ; O=C(O)C(=O)O]

to form the compound of Formula (I), wherein the reaction comprises the following steps:
   (i) to a solution of the compound Formula (A-oxalate hydrate) adding 3-chloropropanoic acid; and
   (ii) to the mixture of (i), adding 1,8-diazabicyclo[5.4.0]undec-7-ene;
to prepare the compound of Formula (I).

Embodiment 2. The method of embodiment 1, wherein step (i) is performed in the presence of potassium carbonate, diisopropylethylamine, and propylphosphonic acid anhydride in dichloromethane.

Embodiment 3. The method of embodiment 1 or 2, wherein step (ii) is performed in dichloromethane.

Embodiment 4. The method of any one of embodiments 1-3, further comprising step (iii): washing the mixture of (ii) with hydrochloride.

Embodiment 5. The method of embodiment 4, further comprising step (iv): dehydrating the organic layer obtained from (iii).

Embodiment 6. The method of embodiment 5, further comprising step (v): adding seeds of compound (I)-HCl to the organic layer of (iv).

Embodiment 7. The method of embodiment 6, further comprising step (vi): adding ethyl acetate to the mixture of (v) at a temperature ranging from 0 to 25° C.

Embodiment 8. The method of embodiment 7, further comprising step (vii): filtering the solid from the mixture of (vi) and adding dichloromethane and sodium bicarbonate to the mixture.

Embodiment 9. The method of embodiment 8, further comprising step (viii): filtering and concentrating the mixture of (vii).

Embodiment 10. The method of any one of embodiments 1-9, wherein the compound of Formula (A-oxalate hydrate) is prepared by reacting a compound of Formula (A):

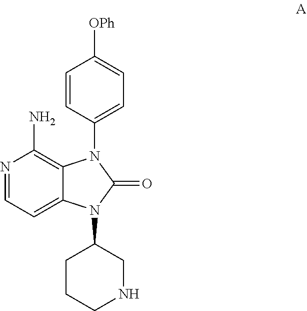 [SMILES: Nc1nccc2c1n(-c1ccc(Oc3ccccc3)cc1)c(=O)n2[C@@H]1CCCNC1]

or a salt thereof, with oxalic acid in a mixture of ethanol and water.

Embodiment 11. A method of preparing a compound of Formula (A-oxalate hydrate):

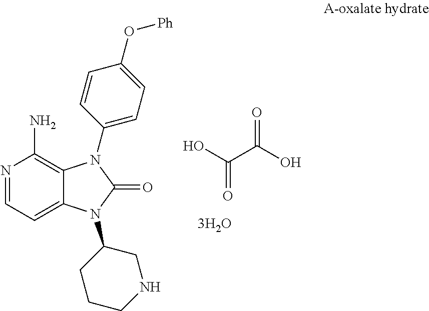 [SMILES: Nc1nccc2c1n(-c1ccc(Oc3ccccc3)cc1)c(=O)n2[C@@H]1CCCNC1 ; O=C(O)C(=O)O]

comprising:
reacting a compound of Formula (A):

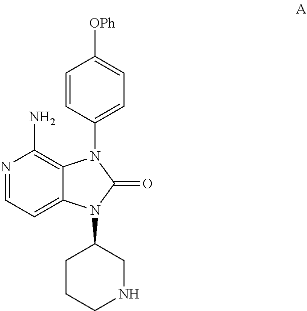 [SMILES: Nc1nccc2c1n(-c1ccc(Oc3ccccc3)cc1)c(=O)n2[C@@H]1CCCNC1]

or a salt thereof, with oxalic acid in a mixture of ethanol and water to form the compound of Formula (A-oxalate hydrate).

Embodiment 12. The method of embodiment 10 or 11, wherein the reaction comprises the addition of crystalline seeds of the compound of Formula (A-oxalate hydrate).

Embodiment 13. The method of embodiment 9, wherein the filtrate of (viii) comprises the compound of Formula (I), or a salt thereof, that is substantially free of an impurity compound of Formula (I)-Im:

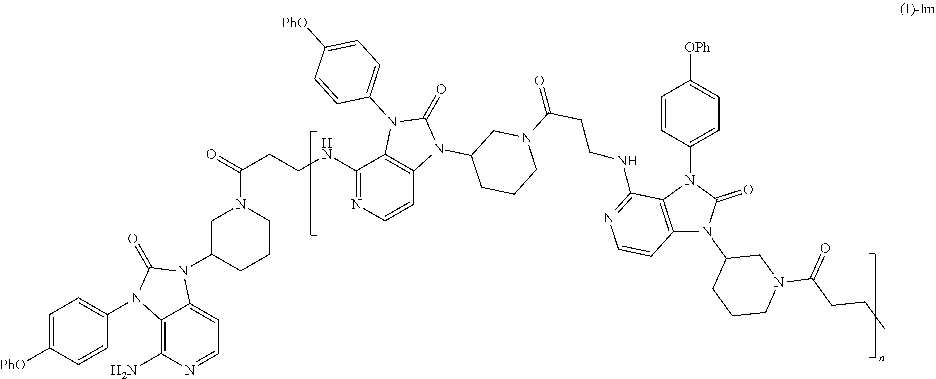

wherein n is equal to or greater than 1.

Embodiment 14. The method of embodiment 9, wherein the filtrate of (viii) yields a product that comprises the compound of Formula (I), or a salt thereof, and an impurity amount ranging from 0 to 1.4%.

Embodiment 15. The method of embodiment 14, wherein the impurity amount ranges from 0 to 0.9%.

Embodiment 16. The method of embodiment 14 or 15, wherein the impurity amount ranges from 0 to 0.4%.

Embodiment 17. The method of any one of embodiments 14-16, wherein the impurity is a compound of Formula (I)-Im:

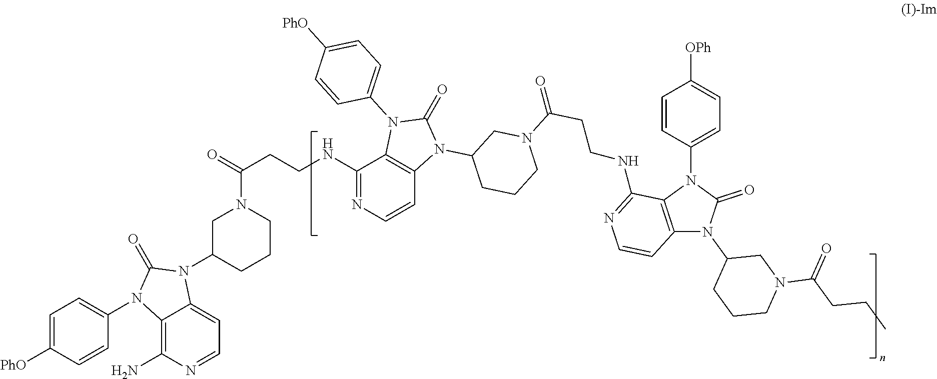

wherein n is equal to or greater than 1.

Embodiment 18. A compound of Formula (I):

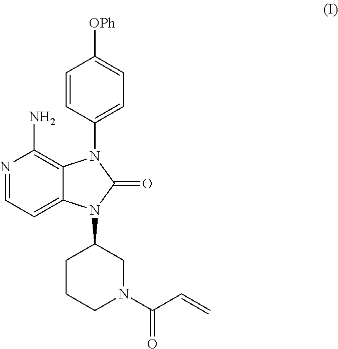 [SMILES: C=CC(=O)N1CCC[C@@H](n2c(=O)n(-c3ccc(Oc4ccccc4)cc3)c3c(N)nccc32)C1]

or a salt thereof, that comprises an impurity in an amount ranging from 0 to 1.4%.

Embodiment 19. The compound of embodiment 18, wherein the impurity amount ranges from 0 to 0.9%.

Embodiment 20. The compound of embodiment 18 or 19, wherein the impurity amount ranges from 0 to 0.4%.

Embodiment 21. A compound of Formula (I):

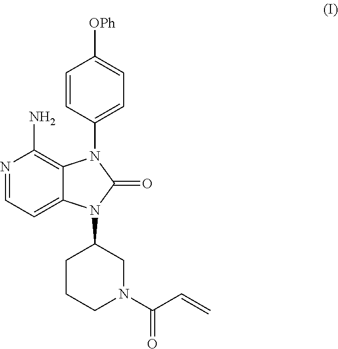 [SMILES: C=CC(=O)N1CCC[C@@H](n2c(=O)n(-c3ccc(Oc4ccccc4)cc3)c3c(N)nccc32)C1]

or a salt thereof, that is substantially free of an impurity compound of Formula (I)-Im:

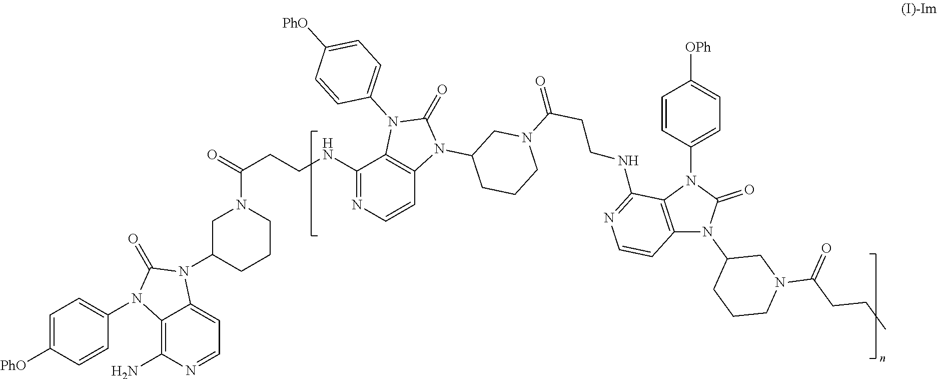

wherein n is equal to or greater than 1.

Embodiment 22. The compound of embodiment 21, wherein the compound of Formula (I), or a salt thereof, comprises the impurity in an amount ranging from 0 to 1.4%.

Embodiment 23. The compound of embodiment 21 or 22, wherein the compound of Formula (I), or a salt thereof, comprises the impurity in an amount ranging from 0 to 0.9%.

Embodiment 24. The compound of any one of embodiments 21-23, wherein the compound of Formula (I), or a salt thereof, comprises the impurity in an amount ranging from 0 to 0.4%.

Embodiment 25. The compound of any one of embodiments 18-20, wherein the impurity is a compound of Formula (I)-Im:

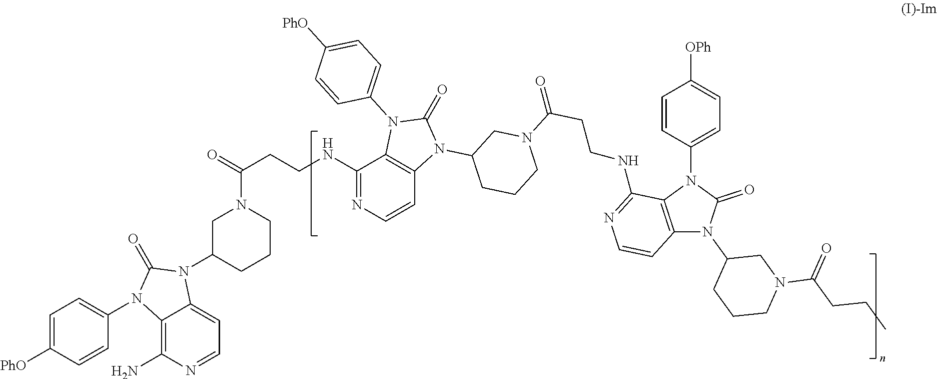

or a salt thereof, wherein n is equal to or greater than 1.

Embodiment 26. A composition comprising a compound of Formula (I) prepared by the method of embodiment 1, wherein the composition is substantially free of an impurity compound of Formula (I)-Im:

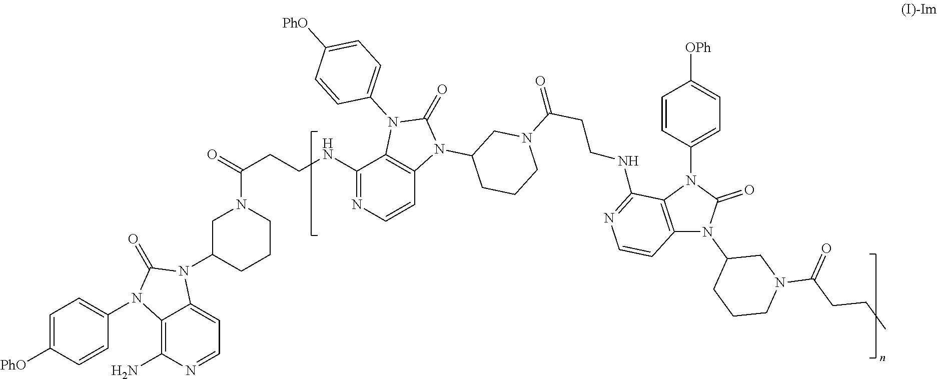

wherein n is equal to or greater than 1.

Embodiment 27. The composition of embodiment 26, wherein the composition comprises at least 97% by weight or by HPLC analysis the compound of Formula (I).

Embodiment 28. The composition of embodiment 26 or 27, wherein the composition comprises an impurity in an amount ranging from 0 to 1.4% by weight or by HPLC analysis.

Embodiment 29. The composition of any one of embodiments 26-28, wherein the impurity amount ranges from 0 to 0.9% by weight or by HPLC analysis.

Embodiment 30. The composition of any one of embodiments 26-29, wherein the impurity amount ranges from 0 to 0.4% by weight or by HPLC analysis.

Embodiment 31. The composition of any one of embodiments 26-30, wherein the impurity is a compound of Formula (I)-Im:

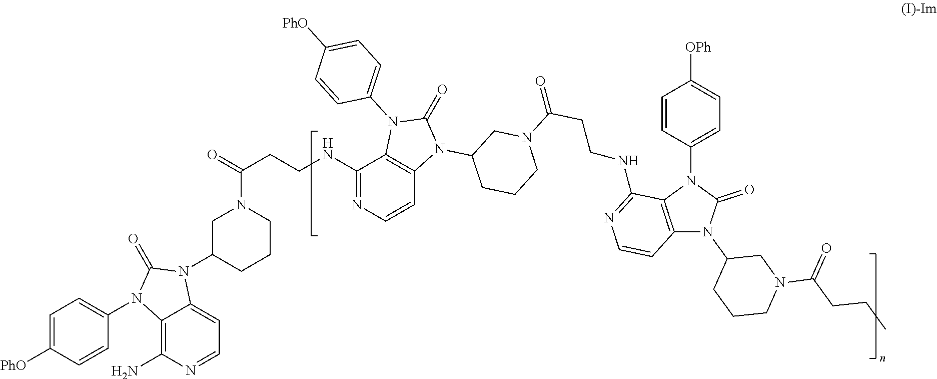

or a salt thereof, wherein n greater than 1.

Embodiment 32. A compound of Formula (I)-Im:

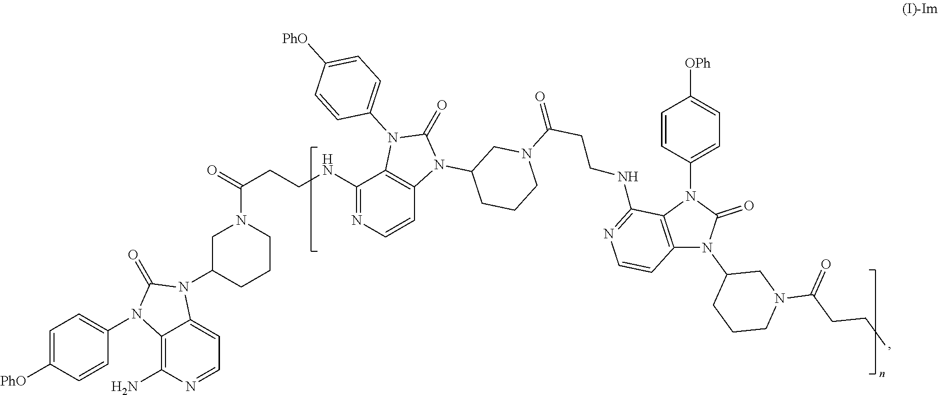

wherein n is equal to or greater than 1.

Embodiment 33. A compound of Formula (A-oxalate hydrate):

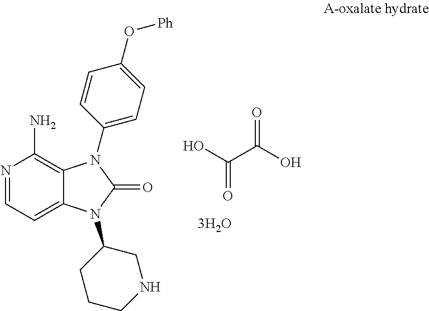 [SMILES: Nc1nccc2c1n(-c1ccc(Oc3ccccc3)cc1)c(=O)n2[C@@H]1CCCNC1 ; O=C(O)C(=O)O]

wherein the compound is about 90% pure.

Embodiment 34. The compound of embodiment 33, wherein the compound is 95% pure.

Embodiment 35. The compound of embodiment 33 or 34, wherein the compound is 98% pure.

Embodiment 36. A crystalline form of a compound of Formula (A-oxalate hydrate):

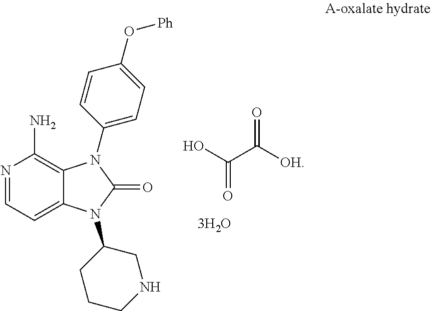 [SMILES: Nc1nccc2c1n(-c1ccc(Oc3ccccc3)cc1)c(=O)n2[C@@H]1CCCNC1 ; O=C(O)C(=O)O]

Embodiment 37. The crystalline form of embodiment 36, having an ORTEP structure that is substantially in accordance with that shown in FIG. 1.

Embodiment 38. The crystalline form of embodiment 36 or 37, wherein the stoichiometry between the compound of Formula (A) and water is 2:6.

What is claimed is:

1. A method of preparing a compound of Formula (I): 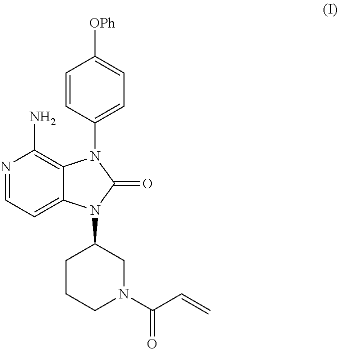 [SMILES: C=CC(=O)N1CCC[C@@H](n2c(=O)n(-c3ccc(Oc4ccccc4)cc3)c3c(N)nccc32)C1]
or a salt thereof, comprising:
reacting a compound of Formula (A-oxalate hydrate): 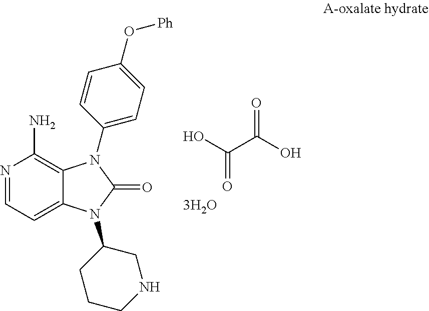 [SMILES: Nc1nccc2c1n(-c1ccc(Oc3ccccc3)cc1)c(=O)n2[C@@H]1CCCNC1 ; O=C(O)C(=O)O]
to form the compound of Formula (I), wherein the reaction comprises the following steps:
(i) to a solution of the compound Formula (A-oxalate hydrate) adding 3-chloropropanoic acid; and
(ii) to the mixture of (i), adding 1,8-diazabicyclo[5.4.0]undec-7-ene;
to prepare the compound of Formula (I).

2. The method of claim 1, wherein step (i) is performed in the presence of potassium carbonate, diisopropylethylamine, and propylphosphonic acid anhydride in dichloromethane.

3. The method of claim 1 or 2, wherein step (ii) is performed in dichloromethane.

4. The method of any one of claims 1-3, further comprising step (iii): washing the mixture of (ii) with hydrochloride.

5. The method of claim 4, further comprising step (iv): dehydrating the organic layer obtained from (iii).

6. The method of claim 5, further comprising step (v): adding seeds of compound (I)-HCl to the organic layer of (iv).

7. The method of claim 6, further comprising step (vi): adding ethyl acetate to the mixture of (v) at a temperature ranging from 0 to 25° C.

8. The method of claim 7, further comprising step (vii): filtering the solid from the mixture of (vi) and adding dichloromethane and sodium bicarbonate to the mixture.

9. The method of claim 8, further comprising step (viii): filtering and concentrating the mixture of (vii).

10. The method of any one of claims 1-9, wherein the compound of Formula (A-oxalate hydrate) is prepared by reacting a compound of Formula (A): 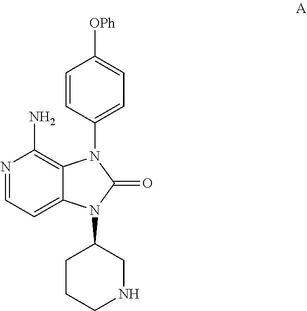 [SMILES: Nc1nccc2c1n(-c1ccc(Oc3ccccc3)cc1)c(=O)n2[C@@H]1CCCNC1]
or a salt thereof, with oxalic acid in a mixture of ethanol and water.

11. A method of preparing a compound of Formula (A-oxalate hydrate): 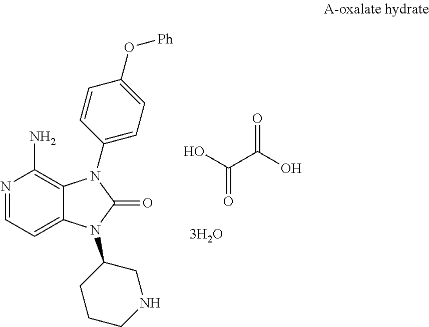 [SMILES: Nc1nccc2c1n(-c1ccc(Oc3ccccc3)cc1)c(=O)n2[C@@H]1CCCNC1 ; O=C(O)C(=O)O]
comprising:
reacting a compound of Formula (A): 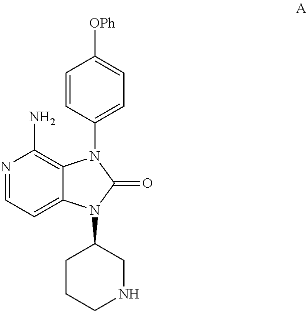 [SMILES: Nc1nccc2c1n(-c1ccc(Oc3ccccc3)cc1)c(=O)n2[C@@H]1CCCNC1]
or a salt thereof, with oxalic acid in a mixture of ethanol and water to form the compound of Formula (A-oxalate hydrate).

12. The method of claim 10 or 11, wherein the reaction comprises the addition of crystalline seeds of the compound of Formula (A-oxalate hydrate).

13. The method of claim 9, wherein the filtrate of (viii) comprises the compound of Formula (I), or a salt thereof, that is substantially free of an impurity compound of Formula (I)-Im: 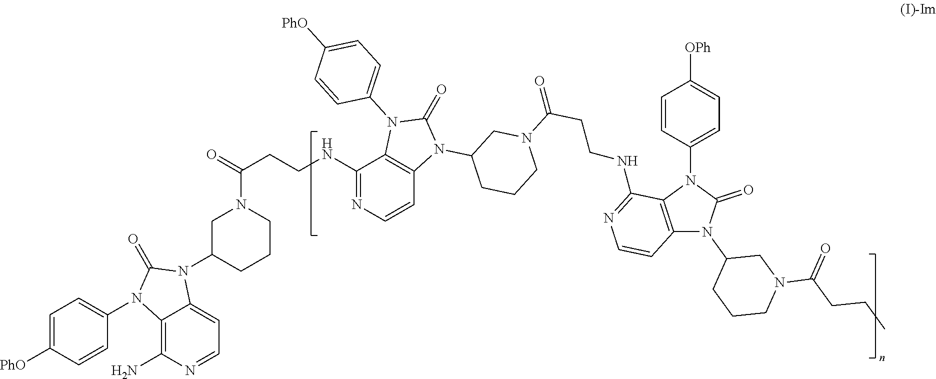
wherein n is equal to or greater than 1.

14. The method of claim 9, wherein the filtrate of (viii) yields a product that comprises the compound of Formula (I), or a salt thereof, and an impurity amount ranging from 0 to 1.4%.

15. The method of claim 14, wherein the impurity amount ranges from 0 to 0.9%.

16. The method of claim 14 or 15, wherein the impurity amount ranges from 0 to 0.4%.

17. The method of any one of claims 14-16, wherein the impurity is a compound of Formula (I)-Im: 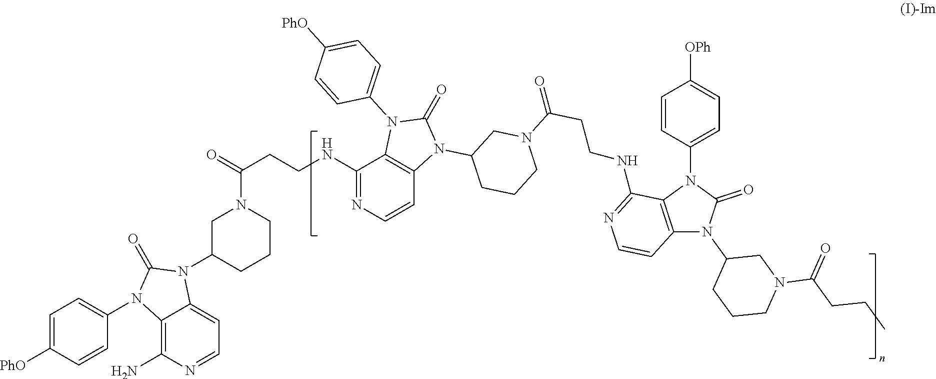
wherein n is equal to or greater than 1.

18. A compound of Formula (I): 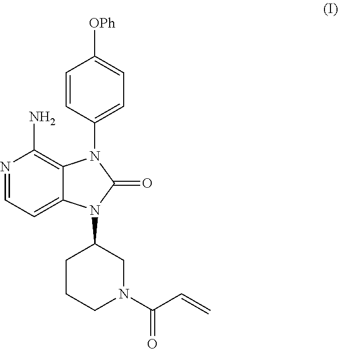 [SMILES: C=CC(=O)N1CCC[C@@H](n2c(=O)n(-c3ccc(Oc4ccccc4)cc3)c3c(N)nccc32)C1]
or a salt thereof, that comprises an impurity in an amount ranging from 0 to 1.4%.

19. The compound of claim 18, wherein the impurity amount ranges from 0 to 0.9%.

20. The compound of claim 18 or 19, wherein the impurity amount ranges from 0 to 0.4%.

21. A compound of Formula (I): 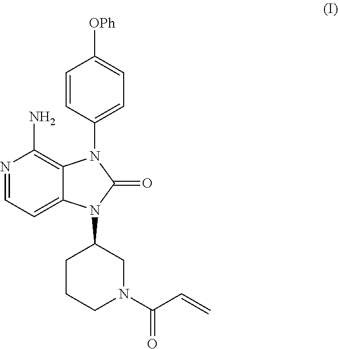 [SMILES: C=CC(=O)N1CCC[C@@H](n2c(=O)n(-c3ccc(Oc4ccccc4)cc3)c3c(N)nccc32)C1]
or a salt thereof, that is substantially free of an impurity compound of Formula (I)-Im: 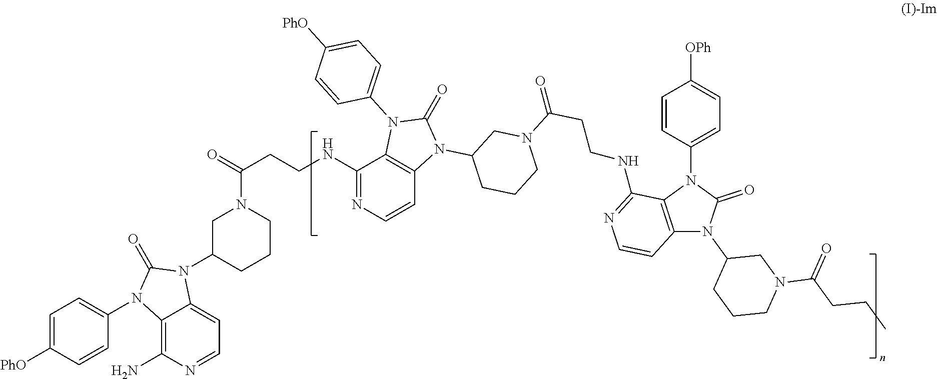
wherein n is equal to or greater than 1.

22. The compound of claim 21, wherein the compound of Formula (I), or a salt thereof, comprises the impurity in an amount ranging from 0 to 1.4%.

23. The compound of claim 21 or 22, wherein the compound of Formula (I), or a salt thereof, comprises the impurity in an amount ranging from 0 to 0.9%.

24. The compound of any one of claims 21-23, wherein the compound of Formula (I), or a salt thereof, comprises the impurity in an amount ranging from 0 to 0.4%.

25. The compound of any one of claims 18-20, wherein the impurity is a compound of Formula (I)-Im: 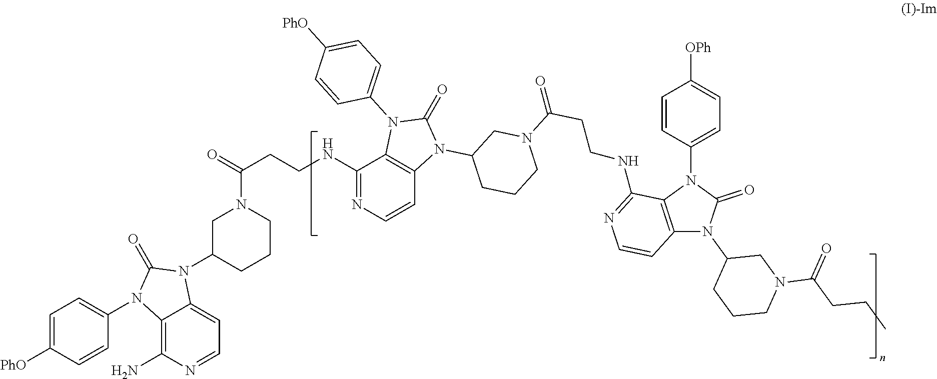
or a salt thereof, wherein n is equal to or greater than 1.

26. A composition comprising a compound of Formula (I) prepared by the method of claim 1, wherein the composition is substantially free of an impurity compound of Formula (I)-Im: 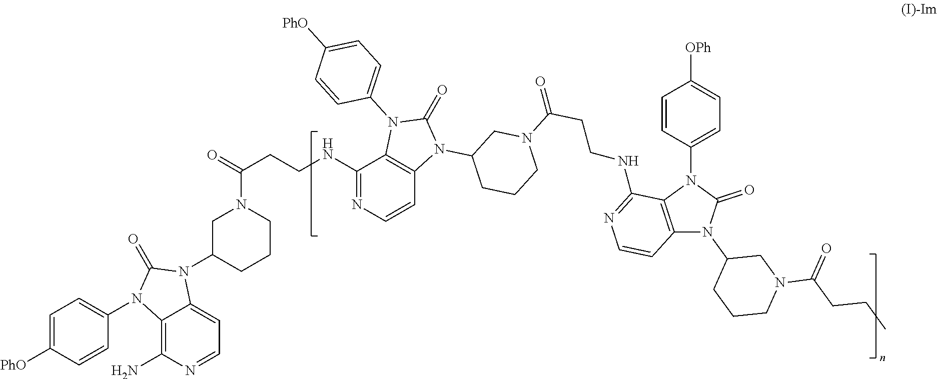
wherein n is equal to or greater than 1.

27. The composition of claim 26, wherein the composition comprises at least 97% by weight or by HPLC analysis the compound of Formula (I).

28. The composition of claim 26 or 27, wherein the composition comprises an impurity in an amount ranging from 0 to 1.4% by weight or by HPLC analysis.

29. The composition of any one of claims 26-28, wherein the impurity amount ranges from 0 to 0.9% by weight or by HPLC analysis.

30. The composition of any one of claims 26-29, wherein the impurity amount ranges from 0 to 0.4% by weight or by HPLC analysis.

31. The composition of any one of claims 26-30, wherein the impurity is a compound of Formula (I)-Im: 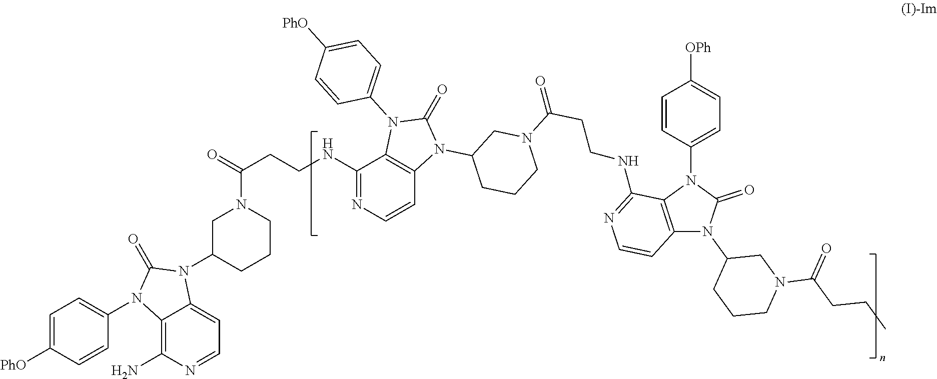
or a salt thereof, wherein n is greater than 1.

32. A compound of Formula (I)-Im: 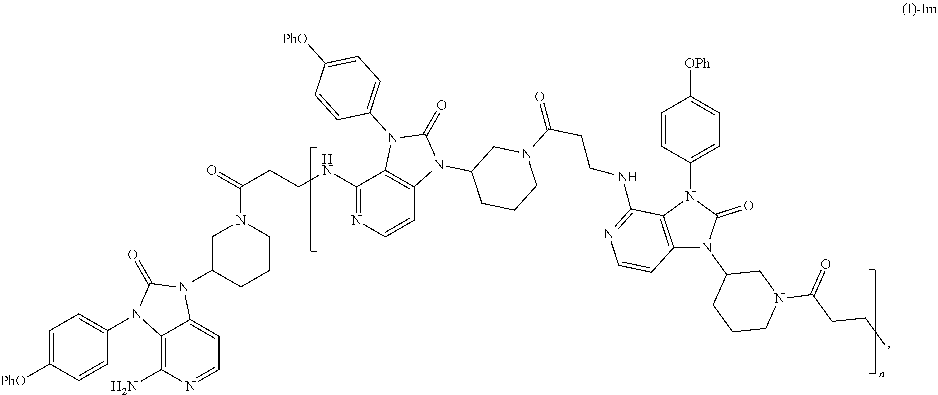
or a salt thereof, wherein n is equal to or greater than 1.

33. A compound of Formula (A-oxalate hydrate): 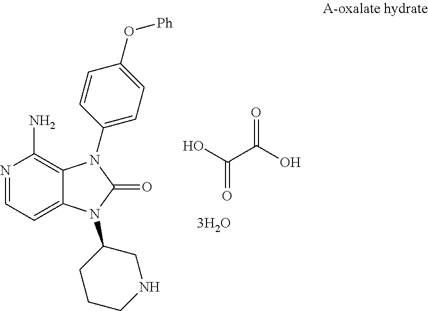 [SMILES: Nc1nccc2c1n(-c1ccc(Oc3ccccc3)cc1)c(=O)n2[C@@H]1CCCNC1 ; O=C(O)C(=O)O]
wherein the compound is about 90% pure.

34. The compound of claim 33, wherein the compound is 95% pure.

35. The compound of claim 33 or 34, wherein the compound is 98% pure.

36. A crystalline form of a compound of Formula (A-oxalate hydrate):

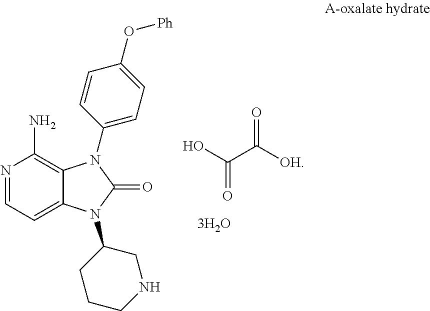 [SMILES: Nc1nccc2c1n(-c1ccc(Oc3ccccc3)cc1)c(=O)n2[C@@H]1CCCNC1 ; O=C(O)C(=O)O]

37. The crystalline form of claim 36, having an ORTEP structure that is substantially in accordance with that shown in FIG. 1.

38. The crystalline form of claim 36 or 37, wherein the stoichiometry between the compound of Formula (A) and water is 2:6.
